(19) United States
(12) Patent Application Publication
(10) Pub. No.: US 2020/0022325 A1
(43) Pub. Date: Jan. 23, 2020

(54) PHYTOHORMONE ENRICHED MICROALGAE METHODS AND COMPOSITIONS

(71) Applicant: HELIAE DEVELOPMENT, LLC, Gilbert, AZ (US)

(72) Inventors: Eneko Ganuza Taberna, Tempe, AZ (US); Danielle Gonzalez, Mesa, AZ (US); Ganapathy Chellappan, Mannheim (DE)

(21) Appl. No.: 16/469,324

(22) PCT Filed: Dec. 14, 2017
(86) PCT No.: PCT/US2017/066365

§ 371 Date: Jun. 13, 2019

Related U.S. Application Data
(60) Provisional application No. 62/434,046, filed on Dec. 14, 2016.

(51) Int. Cl. A01H 4/00; A01H 13/00

(52) U.S. Cl. CPC A01H 4/001; A01H 13/00

(57) ABSTRACT

Methods, systems and compositions for enriching microalgae with concentrations of phytohormones, such as Indole-3-acetic acid, during the culturing process are described. Methods and systems for of enhancing plants through the application of phytohormone enriched microalgae to the plants, and compositions of phytohormone enriched microalgae for the application to plants are also described herein.

### CROSS-REFERENCE TO RELATED APPLICATIONS

This application is a continuation of and claims the benefit of U.S. Provisional Application Ser. No. 62/434,046 filed on Dec. 14, 2016. The entirety of such application is incorporated herein by reference.

### BACKGROUND

Vascular plants utilize a group of molecules that act as chemical messengers for development. These molecules are able to help coordinate growth, stress responses and reproduction by regulating cellular activities, amongst other things, at low concentrations. Such molecules are known as phytohormones. Auxins are a class of phytohormones that are involved with many growth and behavioral processes, including the regulation and inducement of the process of root formation, which vascular plants use to obtain nutrients needed for appropriate development. Phytohormones, in an extracted form, can be used in plant nutrient product formulations as an isolated ingredient. Some microalgae may comprise low levels of phytohormones that are found integrated in the cell,

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Techniques and systems for enriching microalgae with concentrations of phytohormones, by culturing the microalgae with at least one phytohormone precursor, are described. As one exemplary embodiment, microalgae can be cultured with tryptophan to increase the concentration of Indole-3-acetic acid (IAA) in the cell, and increase the relative concentration of IAA in the aqueous fraction of the cell. Additionally, compositions of the phytohormone enriched microalgae for application to plants, and techniques of applying the phytohormone enriched microalgae to plants to enhance at least one plant characteristic are described herein.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

The methods and systems described herein may take physical form in certain parts and arrangements of parts, a preferred embodiment of which will be described in detail in the specification and illustrated in the accompanying drawings which form a part hereof, and wherein:

FIG. 1 illustrates an exemplary block diagram of a system, according to an embodiment.

FIG. 2 illustrates a schematic side view of a system, according to an embodiment.

FIG. 3 illustrates an exemplary block diagram of a system, according to an embodiment.

FIG. 4 illustrates a system, according to an embodiment.

FIG. 5 illustrates a perspective view of an exemplary modular bioreactor system embodiment with modules that can be coupled and decoupled.

FIG. 6 illustrates a perspective view of an exemplary cascading transfer bioreactor system embodiment.

FIG. 7 illustrates a perspective view of an open raceway pond bioreactor embodiment with turning vanes and thrusters.

FIG. 8 shows the chemical relationship tryptophan and Indole-3-acetic acid (IAA).

FIG. 9 shows a comparison of the cell dry weight over time for microalgae cultures treated without and without tryptophan.

FIG. 10 shows a comparison of the cell dry weight over time for microalgae cultures treated without and without tryptophan.

FIG. 11 shows a comparison of the bacteria to microalgae cell ratio over time for microalgae cultures treated without and without tryptophan.

FIG. 12 shows a comparison of the cell dry weight over time for microalgae cultures treated without and without tryptophan.

FIG. 13 shows a comparison of the final IAA concentration for microalgae cultures treated without and without tryptophan.

FIG. 14 shows a comparison of the buds formed on pepper plants treated with microalgae enriched with IAA and non-enriched microalgae.

FIG. 15 shows a comparison of the root dry weight for pepper plants treated with microalgae enriched with IAA and non-enriched microalgae.

FIG. 16 shows a comparison of the shoot dry weight for pepper plants treated with microalgae enriched with IAA and non-enriched microalgae.

FIG. 17 shows a comparison of the tray fruit weight for pepper plants treated with microalgae enriched with IAA and non-enriched microalgae.

FIG. 18 is a flow diagram illustrating an example method for increasing a phytohormone yield in a microalgal culture.

FIG. 19 is a flow diagram illustrating an example alternate embodiment of one or more portions of a method, as described herein.

FIG. 20 is a flow diagram illustrating an example alternate embodiment of one or more portions of a method, as described herein.

FIG. 21 is a diagram illustrating an example composition for treating a plant to enhance a plant characteristic.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

With reference to the drawings, like reference numerals designate identical or corresponding parts throughout the several views. However, the inclusion of like elements in different views does not mean a given embodiment necessarily includes such elements or that all embodiments of the inventive concepts disclosed include such elements. The examples and figures are illustrative only and not meant to limit the inventive concept, which is measured by the scope and spirit of the claims.

The term “microalgae” refers to microscopic single cell organisms such as microalgae, cyanobacteria, algae, diatoms, dinoflagellates, freshwater organisms, marine organisms, or other similar single cell organisms capable of growth in phototrophic, mixotrophic, or heterotrophic culture conditions.

Non-limiting examples of microalgae that can be used in the compositions and methods of the present innovations described herein comprise microalgae in the classes: Eustigmatophyceae, Chlorophyceae, Prasinophyceae, Haptophyceae, Cyanidiophyceae, Prymnesiophyceae, Porphyridiophyceae, Labyrinthulomycetes, Trebouxiophyceae, Bacillariophyceae, and Cyanophyceae. The class Cyanidiophyceae includes species of Galdieria. The class Chlorophyceae includes species of Chlorella, Haematococcus, Scenedesmus, Chlamydomonas, and Micractinium. The class Prymnesiophyceae includes species of Isochrysis and Pavlova. The class Eustigmatophyceae includes species of Nannochloropsis. The class Porphyridiophyceae includes species of Porphyridium. The class Labyrinthulomycetes includes species of Schizochytrium and Aurantiochytrium. The class Prasinophyceae includes species of Tetraselmis. The class Trebouxiophyceae includes species of Chlorella. The class Bacillariophyceae includes species of Phaeodactylum. The class Cyanophyceae includes species of Spirulina.

Non-limiting examples of microalgae genus and species that can be used in the compositions and methods of the present innovations disclosed herein include: Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Aurantiochytrium sp., Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomonas sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Galdieria sp., Gleocapsa sp., Gloeothamnion sp., Haematococcus pluvialis, Hymenomonas sp., Isochrysis aff. galbana, Isochrysis galbana, Lepocinclis, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Porphyridium sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

Taxonomic classification has been in flux for organisms in the genus Schizochytrium. Some organisms previously classified as Schizochytrium have been reclassified as Aurantiochytrium, Thraustochytrium, or Oblongichytrium. See Yokoyama et al. Taxonomic rearrangement of the genus Schizochytrium sensu lato based on morphology, chemotaxonomic characteristics, and 18S rRNA gene phylogeny (Thrausochytriaceae, Labyrinthulomycetes): emendation for Schizochytrium and erection of Aurantiochytrium and Oblongichytrium gen. nov. Mycoscience (2007) 48:199-211. Those of skill in the art will recognize that Schizochytrium, Aurantiochytrium, Thraustochytrium, and Oblongichytrium appear closely related in many taxonomic classification trees for microalgae, and strains and species may be re-classified from time to time. Thus, for references throughout the instant specification for Schizochytrium, it is recognized that microalgae strains in related taxonomic classifications with similar characteristics to Schizochytrium, such as Aurantiochytrium, would reasonably be expected to produce similar results.

In some embodiments, the microalgae may be cultured in phototrophic, mixotrophic, or heterotrophic culture conditions in an aqueous culture medium. The organic carbon sources suitable for growing microalgae mixotrophically or heterotrophically may comprise: acetate, acetic acid, ammonium linoleate, arabinose, arginine, aspartic acid, butyric acid, cellulose, citric acid, ethanol, fructose, fatty acids, galactose, glucose, glycerol, glycine, lactic acid, lactose, maleic acid, maltose, mannose, methanol, molasses, peptone, plant based hydrolyzate, proline, propionic acid, ribose, sacchrose, partial or complete hydrolysates of starch, sucrose, tartaric, TCA-cycle organic acids, thin stillage, urea, industrial waste solutions, yeast extract, and combinations thereof. The organic carbon source may comprise any single source, combination of sources, and dilutions of single sources or combinations of sources. In some embodiments, the microalgae may be cultured in axenic conditions. In some embodiments, the microalgae may be cultured in non-axenic conditions.

In one non-limiting embodiment, the microalgae of the culture in an aqueous culture medium may comprise Chlorella sp. cultured in mixotrophic conditions comprising a culture medium primary comprised of water with trace nutrients (e.g., nitrates, phosphates, vitamins, metals, etc. found in BG-11 recipe [available from UTEX The Culture Collection of Algae at the University of Texas at Austin, Austin, Tex.]), light as an energy source for photosynthesis, and organic carbon (e.g., acetate, acetic acid, etc.) as both an energy source and a source of carbon. In some embodiments, the culture media may comprise the BG-11 media or a media derived from, or including, the BG-11 culture media (e.g., in which additional component(s) are added to the media and/or one or more elements of the media is increased by 5%, 10%, 15%, 20%, 25%, 33%, 50%, or more over unmodified BG-11 media). In some embodiments, the Chlorella may be cultured in non-axenic mixotrophic conditions in the presence of contaminating organisms, such as but not limited to bacteria, or other microbials (e.g., fungi). Additional detail on methods of culturing such microalgae in non-axenic mixotrophic conditions may be found in WO2014/074769A2 (Ganuza, et al.), hereby incorporated by reference.

In some embodiments, by artificially controlling aspects of the microalgae culturing process such as the organic carbon feed (e.g., acetic acid, acetate), oxygen levels, pH, and light, the culturing process differs from the culturing process that microalgae may experience in nature. In addition to controlling various aspects of the culturing process, intervention by human operators or automated systems (e.g., auxostat system(s)) occurs during the non-axenic mixotrophic culturing of microalgae through contamination control methods to prevent the microalgae from being overrun and outcompeted by contaminating organisms (e.g., fungi, bacteria). Contamination control methods for microalgae cultures are known in the art and such suitable contamination control methods for non-axenic mixotrophic microalgae cultures are disclosed in WO2014/074769A2 (Ganuza, et al.), hereby incorporated by reference. By intervening in the microalgae culturing process, the impact of the contaminating microorganisms can be mitigated by suppressing the proliferation of containing organism populations and the effect on the microalgal cells (e.g., lysing, infection, death, clumping). Thus, through artificial control of aspects of the culturing process and intervening in the culturing process with contamination control methods, the microalgae culture produced as a whole and used in the described inventive compositions differs from the culture that results from a microalgae culturing process that occurs in nature.

In some embodiments, during the culturing process the microalgae culture may also comprise cell debris and compounds excreted from the microalgae cells into the culture medium. As an example, the output of the microalgae culturing process can provide one or more active ingredients for a composition that may be applied to plants for improving yield and quality. In one example, this type of composition may be applied without separate addition to, or supplementation of, the composition with other active ingredients that are not found in the mixotrophic microalgae whole cells and accompanying culture medium (e.g., the composition) from the culturing process; additional ingredients such as, but not limited to: microalgae extracts, macroalgae, macroalgae extracts, liquid fertilizers, granular fertilizers, mineral complexes (e.g., calcium, sodium, zinc, manganese, cobalt, silicon), fungi, bacteria, nematodes, protozoa, digestate solids, chemicals (e.g., ethanolamine, borax, boric acid), humic acid, nitrogen and nitrogen derivatives, phosphorus rock, pesticides, herbicides, insecticides, enzymes, plant fiber (e.g., coconut fiber).

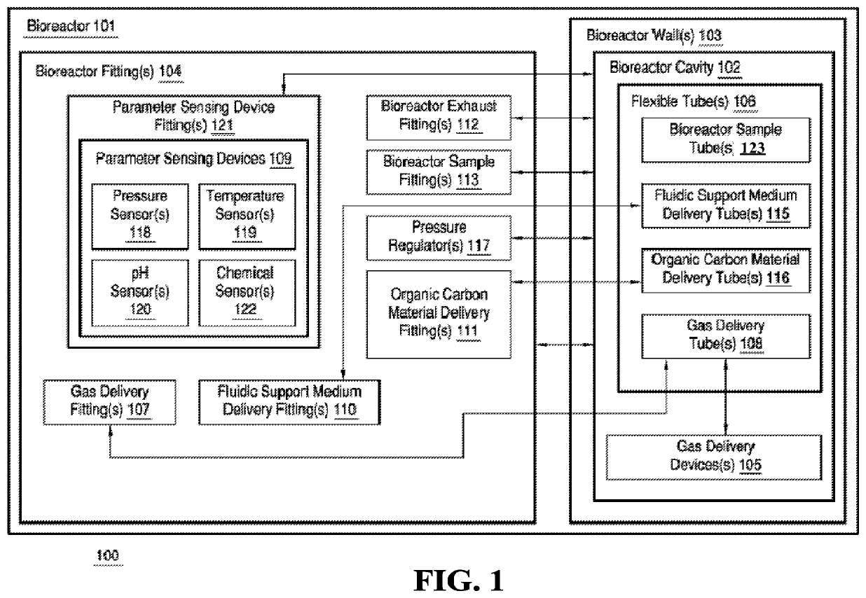

FIG. 1 illustrates an exemplary block diagram of a system 100, according to an embodiment. System 100 is merely exemplary and is not limited to the embodiments presented herein. System 100 can be employed in many different embodiments or examples not specifically depicted or described herein and such adjustments or changes can be selected by one or ordinary skill in the art without departing from the scope of the subject innovation.

System 100 comprises a bioreactor 101 that includes a bioreactor cavity 102 and one or more bioreactor walls 103. Further, bioreactor 101 can include one or more bioreactor fittings 104, one or more gas delivery devices 105, one or more flexible tubes 106, one or more parameter sensing devices 109, and/or one or more pressure regulators 117.

In many embodiments, bioreactor fitting(s) 104 can include one or more gas delivery fittings 107, one or more fluidic support medium delivery fittings 110, one or more organic carbon material delivery fittings 111, one or more bioreactor exhaust fittings 112, one or more bioreactor sample fittings 113, and/or one or more parameter sensing device fittings 121. In these or other embodiments, flexible tube(s) 106 can include one or more gas delivery tubes 123, one or more organic carbon material delivery tubes 116, one or more bioreactor sample tubes 115, and/or one or more fluidic support medium delivery tubes 115. Further, in these or other embodiments, parameter sensing device(s) 109 can include one or more pressure sensors 118, one or more temperature sensors 119, one or more pH sensors 120, and/or one or more chemical sensors 122.

Bioreactor 101 is operable to vitally support (e.g., sustain, grow, nurture, cultivate, among others) one or more organisms (e.g., one or more macroorganisms, one or more microorganisms, and the like). In these or other embodiments, the organism(s) can include one or more autotrophic organisms or one or more heterotrophic organisms. In further embodiments, the organism(s) can comprise one or more mixotrophic organisms. In many embodiments, the organism(s) can comprise one or more phototrophic organisms. In still other embodiments, the organism(s) can comprise one or more genetically modified organisms. In some embodiments, the organism(s) vitally supported by bioreactor 101 can comprise one or more organism(s) of a single type, multiple single organisms of different types, or multiple ones of one or more organisms of different types.

In many embodiments, exemplary microorganism (s) that bioreactor 101 may be implemented to vitally support can include algae (e.g., microalgae), fungi (e.g., mold), and/or cyanobacteria. For example, in many embodiments, bioreactor 101 can be implemented to vitally support multiple types of microalgae such as, but not limited to, microalgae in the classes: Eustigmatophyceae, Chlorophyceae, Prasinophyceae, Haptophyceae, Cyanidiophyceae, Prymnesiophyceae, Porphyridiophyceae, Labyrinthulomycetes, Trebouxiophyceae, Bacillariophyceae, and Cyanophyceae. The class Cyanidiophyceae includes species of Galdieria. The class Chlorophyceae includes species of Chlorella, Haematococcus, Scenedesmus, Chlamydomonas, and Micractinium. The class Prymnesiophyceae includes species of Isochrysis and Pavlova. The class Eustigmatophyceae includes species of Nannochloropsis. The class Porphyridiophyceae includes species of Porphyridium. The class Labyrinthulomycetes includes species of Schizochytrium and Aurantiochytrium. The class Prasinophyceae includes species of Tetraselmis. The class Trebouxiophyceae includes species of Chlorella. The class Bacillariophyceae includes species of Phaeodactylum. The class Cyanophyceae includes species of Spirulina. Further still, in many embodiments, bioreactor 101 can be implemented to vitally support microalgae genus and species as described herein.

Bioreactor cavity 102 can hold (e.g., contain or store) the organism(s) being vitally supported by bioreactor 101, and in many embodiments, can also contain a fluidic support medium configured to hold, and in many embodiments, submerge the organism(s) in a liquid, such as in or part of a culture medium. In many embodiments, the fluidic support medium can comprise a culture medium, and the culture medium can comprise, for example, water. The bioreactor cavity 102 can be at least partially formed and enclosed by one or more bioreactor wall(s) 103. When the bioreactor 101 is implemented with bioreactor fitting(s) 104, bioreactor fitting(s) 104 together with bioreactor wall(s) 103 can fully form and enclose bioreactor cavity 102. Further, as explained in greater detail below, bioreactor wall(s) 103 and one or more of bioreactor fitting(s) 104, as applicable, can be operable to at least partially (e.g., fully) seal the contents of bioreactor cavity 102 (e.g., the organism(s) and/or fluidic support medium) within bioreactor cavity 102. As a result, the bioreactor 101 can maintain conditions mitigating the risk of introducing foreign (e.g., unintended) and/or contaminating organisms to bioreactor cavity 102. In other words, bioreactor 101 can engender the dominance (e.g., proliferation) of certain (e.g., intended) organism(s) being vitally supported at bioreactor 102 over foreign (e g, unintended) and/or contaminating organisms. For example, bioreactor 101 can maintain substantially (e.g., absolutely) axenic conditions in the bioreactor cavity 102.

Bioreactor wall(s) 103 comprise one or more bioreactor wall materials. When bioreactor wall(s) 103 comprise multiple bioreactor walls, two or more of the bioreactor walls can comprise the same bioreactor wall material(s) and/or two or more of the bioreactor walls can comprise different bioreactor wall material(s). In many embodiments, part or all of the bioreactor wall material(s) can comprise (e.g., consist of) one or more flexible materials. In some embodiments, bioreactor 101 can comprise a bag bioreactor.

In these or other embodiments, part or all of the bioreactor wall material(s) (e.g., the flexible material(s)) can comprise one or more partially transparent (e.g., fully transparent) and/or partially translucent (e.g., fully translucent) materials, such as, for example, when bioreactor 101 comprises a photobioreactor (e.g., when the organism(s) comprise phototrophic organism(s)). For example, implementing the bioreactor wall material(s) (e.g., the flexible material(s)) with at least partially transparent or translucent materials can permit light radiation to pass through bioreactor wall(s) 103 to be used as an energy source by the organism(s) contained at bioreactor cavity 102. Still, in some embodiments, bioreactor 101 can vitally support phototrophic organisms when the bioreactor wall material(s) (e.g., the flexible material(s)) of bioreactor wall(s) 103 are opaque, such as, for example, by providing sources of light radiation internal to bioreactor cavity 102. Further, in some embodiments, part or all of the bioreactor wall material(s) (e.g., the flexible material(s)) can comprise one or more selectively partially transparent (e.g., fully transparent) and/or partially translucent (e.g., fully translucent) materials, able to shift from opaque to at least partial transparency (e.g., full transparency) or at least partial translucency (e.g., full translucency).

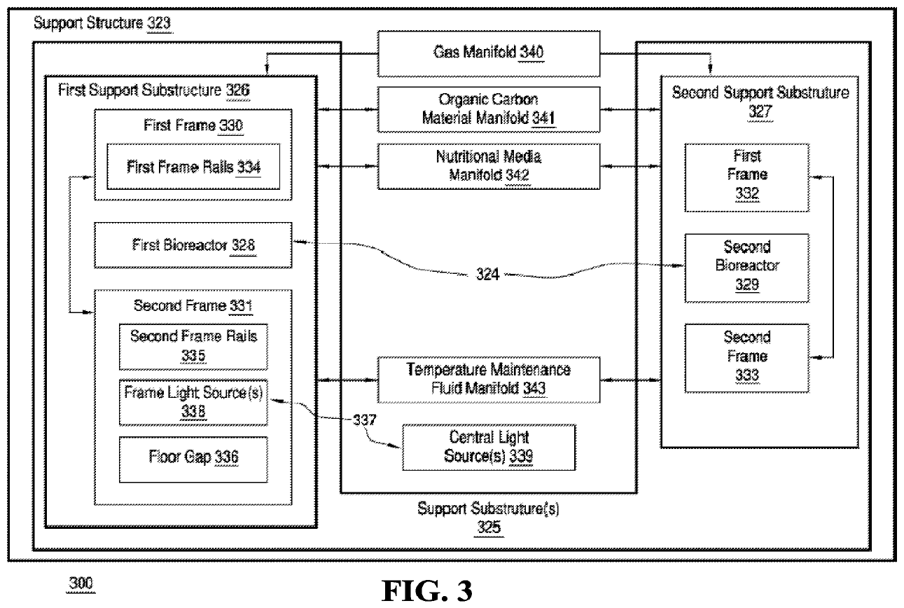

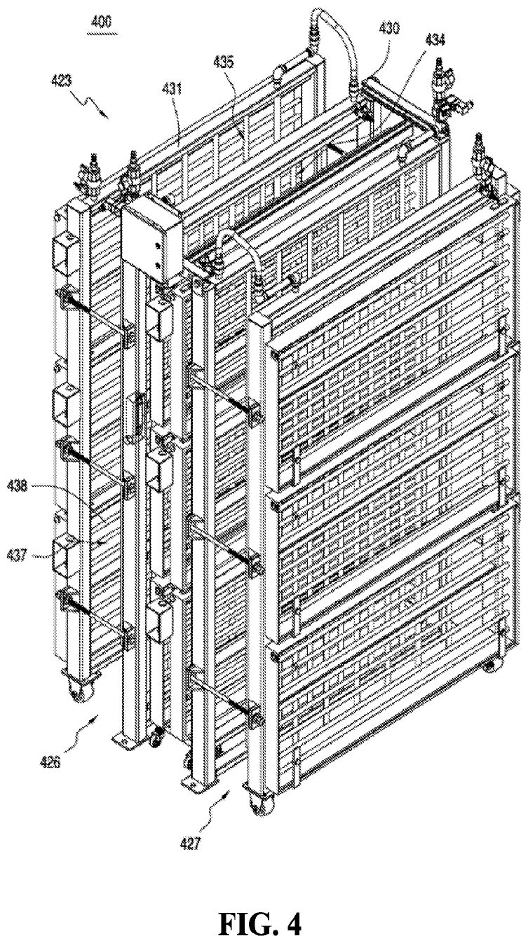

Bioreactor cavity 102 can comprise a cavity volume. The cavity volume of bioreactor cavity 102 can comprise any desirable volume. However, in some embodiments, the cavity volume can be constrained by an available geometry (e.g., the dimensions) of the sheet material(s) used to manufacture bioreactor wall(s) 103. Other factors that can constrain the cavity volume can include a light penetration depth through bioreactor wall(s) 103 and into bioreactor cavity 102 (e.g., when the organism(s) vitally supported by bioreactor 101 are phototrophic organism(s)), a size of an available autoclave for sterilizing bioreactor 101, and/or a size of a support structure implemented to mechanically support bioreactor 101. For example, the support structure can be similar or identical to support structure 323 (shown in FIG. 3) and/or support structure 423 (as shown in FIG. 4).

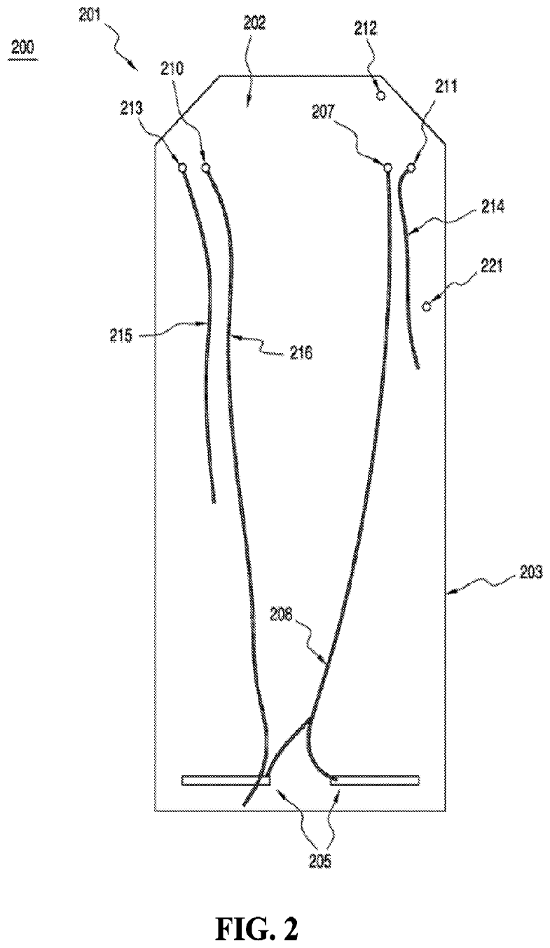

FIG. 2 illustrates a schematic side view of a system 200, according to an embodiment. System 200 is a non-limiting example of system 100 (as shown in FIG. 1). Yet, system 200 of FIG. 2 can be modified or substantially similar to the system 100 of FIG. 1 and such modifications can be selected by one or ordinary skill in the art without departing from the scope of this innovation.

System 200 can comprise bioreactor 201, bioreactor cavity 202, one or more bioreactor walls 203, one or more gas delivery devices 205, one or more gas delivery fittings 207, one or more gas delivery tubes 208, one or more fluidic support medium delivery fittings 210, one or more organic carbon material delivery fittings 211, one or more bioreactor exhaust fittings 212, one or more bioreactor sample fittings 213, one or more organic carbon material delivery tubes 214, one or more bioreactor sample tubes 215, one or more fluidic support medium delivery tubes 216, and one or more parameter sensing device fittings 221. In some embodiments, bioreactor 201 can be similar or identical to bioreactor 101 (as shown in FIG. 1); bioreactor cavity 202 can be similar or identical to bioreactor cavity 102 (as shown in FIG. 1); bioreactor wall(s) 203 can be similar or identical to biore-actor wall(s) 103 (as shown in FIG. 1); gas delivery device(s) 205 can be similar or identical to gas delivery device(s) 105 (as shown in FIG. 1); gas delivery fitting(s) 207 can be similar or identical to gas delivery fitting(s) 107 (as shown in FIG. 1); gas delivery tube(s) 208 can be similar or identical to gas delivery tube(s) 108 (as shown in FIG. 1); fluidic support medium delivery fitting(s) 210 can be similar or identical to fluidic support medium delivery fitting(s) 110 (as shown in FIG. 1); organic carbon material delivery fitting(s) 211 can be similar or identical to organic carbon material delivery fitting(s) 111 (as shown in FIG. 1); bioreactor exhaust fitting(s) 212 can be similar or identical to bioreactor exhaust fitting(s) 112 (as shown in FIG. 1); bioreactor sample fitting(s) 213 can be similar or identical to bioreactor sample fitting(s) 113 (as shown in FIG. 1); organic carbon material delivery tube(s) 214 can be similar or identical to organic carbon material delivery tube(s) 116 (as shown in FIG. 1); bioreactor sample tube(s) 215 can be similar or identical to bioreactor sample tube(s) 123 (as shown in FIG. 1); fluidic support medium delivery tube(s) 216 can be similar or identical to fluidic support medium delivery tube(s) 115 (as shown in FIG. 1); and/or parameter sensing device fitting(s) 221 can be similar or identical to parameter sensing device fitting(s) 121 (as shown in FIG. 1).

Turning ahead now in the drawings, FIG. 3 illustrates an exemplary block diagram of a system 300, according to an embodiment. System 300 is merely exemplary and is not limited to the embodiments presented herein. System 300 can be employed in many different embodiments or examples not specifically depicted or described herein.

System 300 comprises a support structure 323. As explained in greater detail below, support structure 323 is operable to mechanically support one or more bioreactors 324. In these or other embodiments, as also explained in greater detail below, support structure 323 can be operable to maintain a set point temperature of one or more of bioreactor(s) 324. In many embodiments, one or more of bioreactor(s) 324 can be similar or identical to bioreactor 101 (as shown in FIG. 1) and/or bioreactor 201 (as shown in FIG. 2). Accordingly, the term set point temperature can refer to the set point temperature as defined above with respect to system 100 (as shown in FIG. 1). Further, when bioreactor(s) 324 comprise multiple bioreactors, two or more of bioreactor(s) 324 can be similar or identical to each other and/or two or more of bioreactor(s) 324 can be different form each other. For example, the bioreactor wall materials of the bioreactor walls of two or more of bioreactor(s) 324 can be different. In some embodiments, system 300 can comprise one or more of bioreactor(s) 324.

In many embodiments, support structure 323 comprises one or more support substructures 325. Each support substructure of support substructure(s) 325 can mechanically support one bioreactor or more bioreactor(s) 324. In these or other embodiments, each support substructure of support substructure(s) 325 can maintain a set point temperature of one bioreactor of bioreactor(s) 324. In further embodiments, each of support substructure(s) 325 can be similar or identical to each other.

For example, support substructure(s) 325 can comprise a first support substructure 326 and a second support substructure 327. In these embodiments, first support substructure 326 can mechanically support a first bioreactor 328 of bioreactor(s) 324, and second support substructure 327 can mechanically support a second bioreactor 329 of bioreactor(s) 324. Further, first support substructure 326 can comprise a first frame 330 and a second frame 331, and second support substructure 327 can comprise a first frame 332 and a second frame 333. In many embodiments, first frame 330 can be similar or identical to first frame 332, and second frame 331 can be similar or identical to second frame 333. Further, first frame 330 can be similar to second frame 331, and first frame 332 can be similar to second frame 333. It is to be appreciated that the first support substructure 326 can include one or more frames of a first material and the second support substructure 327 can include one or more frames of a second material.

As indicated above, first support substructure 326 can be similar or identical to second support substructure 327. Accordingly, to increase the clarity of the description of system 300 generally, the description of second support substructure 327 is limited so as not to be redundant with respect to first support substructure 326.

In many embodiments, first frame 330 and second frame 331 together can mechanically support first bioreactor 328 in interposition between first frame 330 and second frame 331. That is, bioreactor 328 can be sandwiched between first frame 330 and second frame 331 at a slot formed between first frame 330 and second frame 331. In these or other embodiments, first frame 330 and second frame 331 together can mechanically support first bioreactor 328 in an approximately vertical orientation. Further, first frame 330 and second frame 331 can be oriented approximately parallel to each other. In another embodiment, the first frame 330 and the second frame 331 can be perpendicular to one another.

In many embodiments, second frame 331 can be selectively moveable relative to first frame 330 so that the volume of the slot formed between first frame 330 and second frame 331 can be adjusted. For example, second frame 331 can be supported by one or more wheels permitting second frame 331 to be rolled closer to or further from first frame 330. Meanwhile, in these or other embodiments, second frame 331 can be coupled to first frame 330 by one or more adjustable coupling mechanisms. The adjustable coupling mechanism(s) can hold second frame 331 in a desired position relative to first frame 330 while being adjustable so that the position can be changed when desirable. In implementation, the adjustable coupling mechanism (s) can comprise one or more threaded screws extending between first frame 330 and second frame 331, such as, for example, in a direction orthogonal to first frame 330 and second frame 331. Turning the threaded screws can cause second frame 331 to move (e.g., on the wheel(s)) relative to first frame 330.

Meanwhile, in some embodiments, first frame 330 can be operable to maintain a set point temperature of first bioreactor 328 when first bioreactor 328 is operating to vitally support one or more organisms and when support structure 300 (e.g., first support substructure 326, first frame 330, and/or second frame 331) is mechanically supporting first bioreactor 328. In these or other embodiments, second frame 331 can be operable to maintain the set point temperature of first bioreactor 328 when first bioreactor 328 is operating to vitally support the organism(s) and when support structure 300 (e.g., second support substructure 327, first frame 330, and/or second frame 331) is mechanically supporting first bioreactor 328.

As indicated above, in many embodiments, second frame 331 can be similar or identical to first frame 330. Accordingly, second frame 331 can comprise multiple second frame rails 335. Meanwhile, second frame rails 335 can be similar or identical to first frame rails 334. In some embodiments, the hollow conduits of first frame rails 334 can be coupled to hollow conduits of 335. In these embodiments, the hollow conduits of first frame rails 334 and second frame rails 335 can receive the temperature maintenance fluid from the same source. However, in these or other embodiments, the hollow conduits of first frame rails 334 and the hollow conduits of second frame rails 335 can receive the temperature maintenance fluid from different sources.

In many embodiments, first support substructure 326 comprises a floor gap 336. Floor gap 336 can be located underneath one of first frame 330 or second frame 331. Floor gap 336 can permit first bioreactor 328 to bulge into floor gap 336 past first support substructure 326 when first support substructure 326 is mechanically supporting first bioreactor 328. Permitting first bioreactor 328 to bulge into floor gap 336 can relieve stress from first bioreactor 328. For example, in many embodiments, bioreactor(s) 324 can experience the greatest amount of stress at their base(s) when being mechanically supported in a vertical position, such as, for example, by support structure 323. In these embodiments, permitting first bioreactor 328 to bulge into floor gap 336 such that first support substructure 326 is not restraining first bioreactor 328 at floor gap 336 can relieve more stress from first bioreactor 328 than constraining all of first bioreactor 328 at both sides with first frame 330 and second frame 331, even if first frame 330 and second frame 331 are reinforced.

System 300 (e.g., support structure 323) can comprise one or more light sources 337. Light source(s) 337 can be operable to illuminate the organism(s) being vitally supported at bioreactor(s) 324. In many embodiments, second frame 331 can comprise and/or mechanically support one or more frame light source(s) 338 of light source(s) 337. Meanwhile, system 300 (e.g., support structure 323) can comprise one or more central light source(s) 339. In these or other embodiments, support substructure(s) 325 (e.g., first support substructure 326 and second support substructure 327) can be mirrored about a central vertical plane of support structure 323. Accordingly, central light source(s) 339 can be interpositioned between first support substructure 326 and second support substructure 327 so that first bioreactor 328 and second bioreactor 329 each can receive light from central light source(s) 339.

In implementation, light source(s) 337 (e.g., frame light source(s) 338 and/or central light source(s) 339) can comprise one or more banks of light bulbs and/or light emitting diodes. In some embodiments, light source(s) 337 (e.g., the light bulbs and/or light emitting diodes) can emit one or more wavelengths of light, as desirable for the particular organism(s) being vitally supported by bioreactor(s) 324.

In some embodiments, the one or more light sources 337 may be provided on one side of the bioreactors 324, and a second side of the bioreactors 324 may have no lighting devices or may have the panels with light sources pivoted open. In one non-limiting exemplary embodiment, a system 300 can include light sources 337 on a first side and an open second side to gather natural light.

Advantageously, because each support substructure of support substructure(s) 325 can maintain a set point temperature of different ones of bioreactor(s) 324, each of bioreactor(s) 324 can be maintained at a set point temperature independently of each other. For example, when bioreactor(s) 324 are vitally supporting different types of organism(s), bioreactor(s) 324 can comprise different set point temperatures. Nonetheless, in many embodiments, bioreactor(s) 324 can comprise the same set point temperatures.

Meanwhile, in many embodiments, system 300 can comprise gas manifold 340, organic carbon material manifold 341, nutritional media manifold 342, and/or temperature maintenance fluid manifold 343. Gas manifold 340 can be operable to provide gas to one or more gas delivery fittings of bioreactor(s) 324. The gas delivery fitting(s) can be similar or identical to gas delivery fitting(s) 107 (as shown in FIG. 1) and/or gas delivery fitting(s) 207 (as shown in FIG. 2). Further, organic carbon material manifold 341 can be operable to deliver organic carbon material to one or more organic carbon material delivery fittings of bioreactor(s) 324. The organic carbon material delivery fitting(s) can be similar or identical to organic carbon material delivery fitting(s) 111 (as shown in FIG. 1) and/or organic carbon material delivery fitting(s) 211 (as shown in FIG. 2). Further still, nutritional media manifold 342 can be operable to provide nutritional media to one or more fluidic support medium delivery fittings of bioreactor(s) 324. The fluidic support medium delivery fitting(s) can be similar or identical to fluidic support medium delivery fitting(s) 110 (as shown in FIG. 1) and/or fluidic support medium delivery fitting(s) 210 (as shown in FIG. 2). Meanwhile, temperature maintenance fluid manifold can be configured to provide the temperature maintenance fluid to the hollow conduits of first frame 330 and/or second frame 331.

Gas manifold 340, organic carbon material manifold 341, nutritional media manifold 342, and/or temperature maintenance fluid manifold 343 each can comprise one or more tubes, one or more valves, one or more gaskets, one or more reservoirs, one or more pumps, and/or control logic (e.g., one or more computer processors, one or more transitory memory storage modules, and/or one or more non-transitory memory storage modules) configured to perform their respective functions. In these embodiments, the control logic can communicate with one or more parameter sensing devices of bioreactor(s) 324 to determine when to perform their respective functions (i.e., according to the needs of the organism(s) being vitally supported by bioreactor(s) 324). The parameter sensing device(s) can be similar or identical to parameter sensing device(s) 109 (as shown in FIG. 1).

Turning to the next drawing, FIG. 4 illustrates a system 400, according to an embodiment. System 400 is a non-limiting example of system 300 (as shown in FIG. 3). Yet, system 400 of FIG. 4 can be modified or substantially similar to the system 300 of FIG. 3 and such modifications can be selected by one or ordinary skill in the art without departing from the scope of this innovation.

System 400 can comprise support structure 423, first support substructure 426, second support substructure 427, first frame 430, second frame 431, first frame rails 434, second frame rails 435, and one or more light source(s) 437. In these embodiments, light source(s) 437 can comprise one or more frame light sources 438. In many embodiments, support structure 423 can be similar or identical to support structure 323 (as shown in FIG. 3); first support substructure 426 can be similar or identical to first support substructure 326 (as shown in FIG. 3); second support substructure 427 can be similar or identical to second support substructure 327 (as shown in FIG. 3); first frame 430 can be similar or identical to first frame 330 (as shown in FIG. 3); second frame 431 can be similar or identical to second frame 331 (as shown in FIG. 3); first frame rails 434 can be similar or identical to first frame rails 334 (as shown in FIG. 3); second frame rails 435 can be similar or identical to second frame rails 335 (as shown in FIG. 3); and/or light source(s) 437 can be similar or identical to light source(s) 337 (as shown in FIG. 3). Further, frame light source(s) 438 can be similar or identical to frame light source(s) 338.

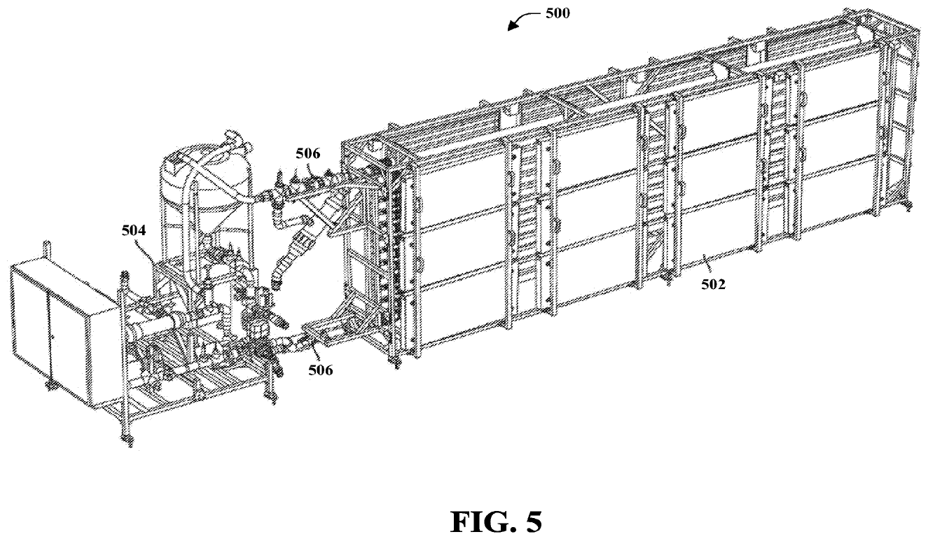

FIG. 5 illustrates an embodiment of a modular bioreactor system 500. In one embodiment, a self-contained bioreactor system for culturing microorganisms in an aqueous medium comprises a modular bioreactor system. The modular bioreactor system comprises a plurality of modular components which may be easily coupled together into a functioning system and decoupled for repair, replacement, upgrading, shipping, cleaning, or reconfiguration. The interchangeability of the modular components allows components of a bioreactor system to be easily transported and assembled at multiple locations, as well as to change the capacity of the bioreactor system or change the functionality of the bioreactor system. Each module is a standalone unit that may be interchanged with other modular bioreactor systems for different configurations, providing the benefit of flexibility over conventional single configuration integrated bioreactor systems.

In some embodiments, the modular components may be decoupled when the modular bioreactor system contains an aqueous culture of microorganisms, while maintaining isolated volumes of the aqueous microorganism culture in the various individual modular components without exposing the culture of microorganisms to the environment or outside contamination. With the ability to maintain an isolated volume of the aqueous culture, modules may be interchanged in the event of equipment malfunction without necessitating harvest or enduring a complete loss of the microorganism culture. Additionally, an isolated volume of the aqueous microorganism culture may be transported to different locations for different operations, such as growth, product maturation (e.g., lipid accumulation, pigment accumulation), harvest, dewatering, etc. The modular components may couple and decouple from each other using pipe or tubular quick connect couplers which may be quickly coupled by hand to allow fluid communication between modular components and quickly decoupled in a manner which also self-seals any fluid communication, effectively sealing an isolated volume of the aqueous culture in each modular component. The quick connect couplers may comprise fluid conduit couplers known in the art such as, but not limited to, cam lock couplers.

A non-limiting exemplary embodiment of a modular bioreactor system 500 is shown in FIG. 5. FIG. 5 shows a modular bioreactor system 500 with a bioreactor module 502, cleaning module 504, and pump and control module 506 coupled together in fluid communication. It is to be appreciated that the modular bioreactor system 500 with a bioreactor module 502, cleaning module 504, and pump and control module 506 can be decoupled from each other. As an example, one or more couplers between the modules may comprise quick connection couplers such as, but not limited to cam lock couplers, capable of self-sealing an isolated volume of an aqueous culture medium in each individual module. In some embodiments of the modular bioreactor system 500, the couplers may comprise traditional couplers such as, but not limited to, threaded connections or bolted together flange connections.

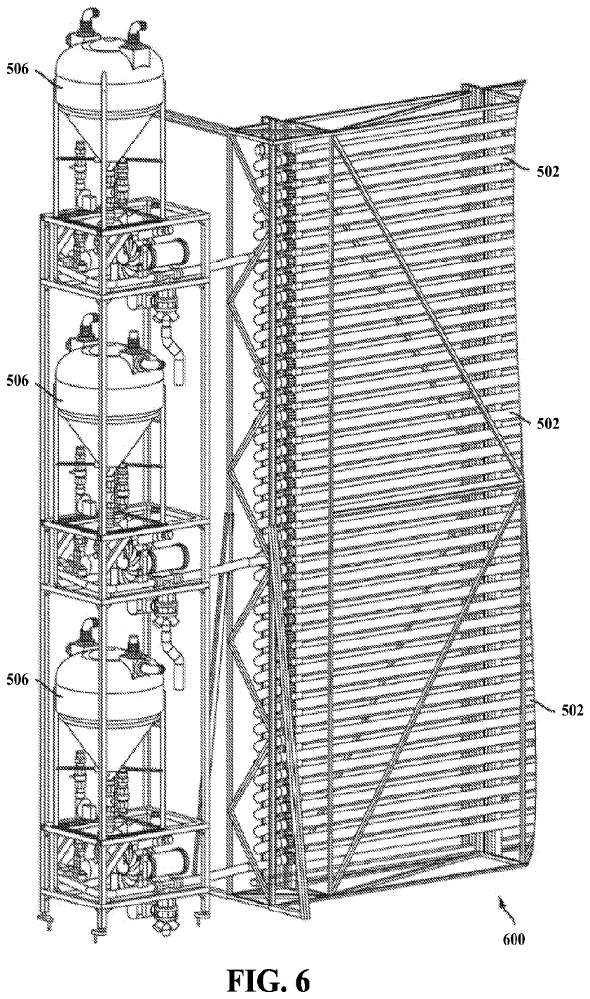

FIG. 6 illustrates a non-limiting exemplary embodiment of a cascading transfer bioreactor system 600 with multiple bioreactor modules 502 and multiple pump and control modules 506. The cascading transfer bioreactor system 600 can include modular bioreactors may be used as a production platform, as a seed reactor platform, or a combination of both. The cascading transfer bioreactor system 600 may be used in a system that connects the seed production with one or more larger volume downstream production reactors. The cascading transfer bioreactor system 600 may be partially or fully harvested to inoculate a larger seed reactor. The cascading transfer bioreactor system 600 may be used as a finishing step for the production of products that require a two-step growth process to produce pigments or other high value products.

In an alternate embodiment, the cascading transfer bioreactor system 600 may comprise culture tube segments that have different diameters, where a small diameter is used for a preferentially phototrophic section while a larger tubular diameter is used for a preferably mixotrophic section. The segments with different culture tube diameters may be interleaved and connected in a way to enhance turbulence or mixing in the system without the use of a high Reynolds numbers such that the overall system pressure drop is reduced.

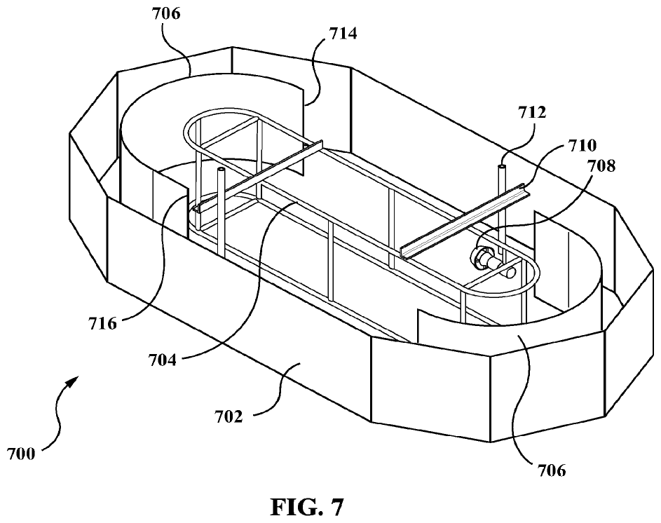

Turning to FIG. 7, a non-limiting embodiment of the open raceway pond bioreactor 700 is illustrated. The open raceway pond bioreactor 700 comprises an outer wall 702, center wall 704, arched turning vanes 706, submerged thrusters 708, support structure 710 (horizontal), and 712 (vertical). The outer wall 702 and the center wall 704 form the boundaries of the straight away portions and U-bend portions of the bioreactor 700. The center wall 704 is shown as a frame for viewing purposes, but in practice panels are inserted into open sections of the frame or a liner placed over the frame to form a solid center wall surface. Also, the outer wall 702 of the bioreactor 700 is depicted as multiple straight segments connected at angles to form the curved portion of the U-bend, but the outer wall 702 may also form a continuous curve or arc.

The arched turning vanes 706 can have an asymmetrical shape having a first end 714 of the turning vane at the beginning of the U-bend portion and a second end 716 extending past the U-bend portion into the straight away portion. The flow path of the culture in the open raceway pond bioreactor 700 would be counter clockwise, with the culture encountering first end 714 of the turning vane first, second end 716 of the turning vane second, and then the submerged thruster 708 when traveling through the U-bend portion and into the straight away portion. The arched turning vanes 706 are also shown in to be at least as tall as the center wall 704, to allow a portion of the arched turning vanes 706 to protrude from the culture volume when operating.

In one aspect, it is known in the art that microalgae cells may be genetically modified to increase the production of certain desired products, such as phytohormones. As described herein, one or more techniques may be devised for increasing the production of phytohormones in microalgae without using genetic modification. Techniques can provide for synthesis and accumulation of phytohormones in the microalgae cell, resulting in an enrichment of phytohormones in the microalgae cell greater than that which may be found in nature. In this aspect, similar to plants, microalgae cultured in the presence of phytohormones have also been shown to benefit in the form of increased lipids and growth. However, existing literature has shown that merely adding phytohormones to the culture medium does not appear to translate into an accumulation of the phytohormones by the microalgae cells.

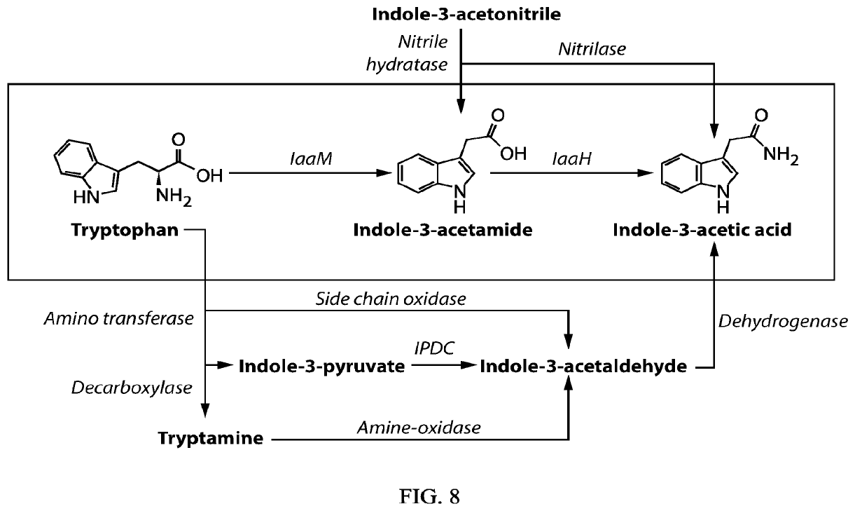

In this aspect, as described herein, a method has been developed that uses a phytohormone precursor as a treatment to a live microalgae culture to trigger biosynthesis of the phytohormone in the microalgae cells. The resulting cultured microalgae express an increase in the accumulation of phytohormones in the microalgae cells. For example, as shown in FIG. 8, L-Tryptophan is a precursor for biosynthesizing the phytohormone Indole-3-acetic acid (IAA), which is a common plant hormone found in a class of hormones called auxins. As an example, auxins are often used in plant nutrient product formulations to produce desired results, such as specialized or target development and growth. In one implementation, treating a microalgae culture with an effective amount of tryptophan resulted in a surprising increase in the concentration of IAA in the microalgae cell, without noticeable negative effects on the typical growth and development of the microalgae.

The techniques and systems, described herein, detail merely one embodiment of the inventive concept, which results in increased production IAA, as a non-limiting example of an auxin class phytohormone. It should be understood that IAA is merely used as an example to describe the inventive concept, and this should not be interpreted in any way as limiting to the techniques and systems described. The inventive method and compositions are intended to be used with phytohormones generally and with known equivalents to phytohormones, including, but not limited to, other auxin class phytohormones, such as: 4-Chloroindole-3-acetic acid (4-CI-IAA); 2-phenylacetic acid (PAA); indole-3-butyric acid (IBA); and indole-3-propionic acid (IPA). Such scope and embodiments are encompassed by the innovative concepts described herein. Further, the term “microalgae” refers to microscopic single cell organisms such as microalgae, cyanobacteria, algae, diatoms, dinoflagellates, freshwater organisms, marine organisms, or other similar single cell organisms capable of growth in phototrophic, mixotrophic, or heterotrophic culture conditions.

In one implementation, in this aspect, in order to increase the concentration of phytohormones in the microalgae cells yield, a precursor to the target phytohormone can be added to a culture of live microalgae cells during the culturing process. In this implementation, the addition of the appropriate phytohormone precursor, in the appropriate conditions, can facilitate biosynthesis of the target phytohormone by the microalgae cells, resulting in a phytohormone enriched microalgae cell. In some embodiments, the precursor may be tryptophan and the target phytohormone may be IAA. In some embodiments, the tryptophan may be L-tryptophan. In some embodiments, the concentration of tryptophan added to a microalgae culture may be in the range of 10-500 mg/L. In some embodiments, the concentration of tryptophan added to a microalgae culture may be in the range of 50-75 mg/L. In some embodiments, the concentration of tryptophan added to a microalgae culture may be in the range of 75-100 mg/L. In some embodiments, the concentration of tryptophan added to a microalgae culture may be in the range of 100-150 mg/L. In some embodiments, the concentration of tryptophan added to a microalgae culture may be in the range of 150-200 mg/L. In some embodiments, the concentration of tryptophan added to a microalgae culture may be in the range of 100-200 mg/L. In some embodiments, the concentration of tryptophan added to a microalgae culture may be in the range of 200-250 mg/L. In some embodiments, the concentration of tryptophan added to a microalgae culture may be in the range of 250-300 mg/L. In some embodiments, the concentration of tryptophan added to a microalgae culture may be in the range of 300-400 mg/L. In some embodiments, the concentration of tryptophan added to a microalgae culture may be in the range of 400-500 mg/L.

In some embodiments, the concentration of tryptophan added to a microalgae culture may be at least 50 mg/L. In some embodiments, the concentration of tryptophan added to a microalgae culture may be at least 75 mg/L. In some embodiments, the concentration of tryptophan added to a microalgae culture may be at least 100 mg/L. In some embodiments, the concentration of tryptophan added to a microalgae culture may be at least 150 mg/L. In some embodiments, the concentration of tryptophan added to a microalgae culture may be at least 200 mg/L. In some embodiments, the concentration of tryptophan added to a microalgae culture may be at least 250 mg/L. In some embodiments, the concentration of tryptophan added to a microalgae culture may be at least 300 mg/L. In some embodiments, the concentration of tryptophan added to a microalgae culture may be at least 400 mg/L. In some embodiments, the concentration of tryptophan added to a microalgae culture may be at least 500 mg/L.

In some embodiments, the concentration of tryptophan added to a microalgae culture may be less than 500 mg/L. In some embodiments, the concentration of tryptophan added to a microalgae culture may be less than 400 mg/L. In some embodiments, the concentration of tryptophan added to a microalgae culture may be less than 300 mg/L. In some embodiments, the concentration of tryptophan added to a microalgae culture may be less than 250 mg/L. In some embodiments, the concentration of tryptophan added to a microalgae culture may be less than 200 mg/L. In some embodiments, the concentration of tryptophan added to a microalgae culture may be less than 150 mg/L. In some embodiments, the concentration of tryptophan added to a microalgae culture may be less than 100 mg/L. In some embodiments, the concentration of tryptophan added to a microalgae culture may be less than 75 mg/L. In some embodiments, the concentration of tryptophan added to a microalgae culture may be less than 50 mg/L.

In some embodiments, the IAA enriched microalgae cells may comprise a concentration in the range of 0.01-1.0 mg IAA/L. In some embodiments, the IAA enriched microalgae cells may comprise a concentration in the range of 0.01-0.05 mg IAA/L. In some embodiments, the IAA enriched microalgae cells may comprise a concentration in the range of 0.05-0.10 mg IAA/L. In some embodiments, the IAA enriched microalgae cells may comprise a concentration in the range of 0.10-0.15 mg IAA/L. In some embodiments, the IAA enriched microalgae cells may comprise a concentration in the range of 0.10-0.20 mg IAA/L. In some embodiments, the IAA enriched microalgae cells may comprise a concentration in the range of 0.20-0.25 mg IAA/L. In some embodiments, the IAA enriched microalgae cells may comprise a concentration in the range of 0.25-0.30 mg IAA/L. In some embodiments, the IAA enriched microalgae cells may comprise a concentration in the range of 0.30-0.40 mg IAA/L. In some embodiments, the IAA enriched microalgae cells may comprise a concentration in the range of 0.40-0.50 mg IAA/L. In some embodiments, the IAA enriched microalgae cells may comprise a concentration in the range of 0.50-0.60 mg IAA/L. In some embodiments, the IAA enriched microalgae cells may comprise a concentration in the range of 0.60-0.70 mg IAA/L. In some embodiments, the IAA enriched microalgae cells may comprise a concentration in the range of 0.70-0.80 mg IAA/L. In some embodiments, the IAA enriched microalgae cells may comprise a concentration in the range of 0.80-0.90 mg IAA/L. In some embodiments, the IAA enriched microalgae cells may comprise a concentration in the range of 0.90-1.0 mg IAA/L.

In some embodiments, the amount of phytohormones in microalgae cells treated with a precursor during the culturing process may increase by a multiple in the range of 100-5,000%. In some embodiments, the amount of phytohormones in microalgae cells treated with a precursor during the culturing process may increase by a multiple in the range of 100-200%. In some embodiments, the amount of phytohormones in microalgae cells treated with a precursor during the culturing process may increase by a multiple in the range of 200-500%. In some embodiments, the amount of phytohormones in microalgae cells treated with a precursor during the culturing process may increase by a multiple in the range of 500-1,000%. In some embodiments, the amount of phytohormones in microalgae cells treated with a precursor during the culturing process may increase by a multiple in the range of 1,000-2,000%. In some embodiments, the amount of phytohormones in microalgae cells treated with a precursor during the culturing process may increase by a multiple in the range of 2,000-3,000%. In some embodiments, the amount of phytohormones in microalgae cells treated with a precursor during the culturing process may increase by a multiple in the range of 3,000-4,000%. In some embodiments, the amount of phytohormones in microalgae cells treated with a precursor during the culturing process may increase by a multiple in the range of 4,000-5,000%.

In some embodiments, the biosynthesized phytohormones may be primarily disposed in the solid fraction of the microalgae cell. In some embodiments, the biosynthesized phytohormones may be primarily disposed in the aqueous fraction of the microalgae cell. In some embodiments, the biosynthesized phytohormones disposed in the aqueous fraction may comprise 30-80%. In some embodiments, the biosynthesized phytohormones disposed in the aqueous fraction may comprise 30-40%. In some embodiments, the biosynthesized phytohormones disposed in the aqueous fraction may comprise 40-50%. In some embodiments, the biosynthesized phytohormones disposed in the aqueous fraction may comprise 50-60%. In some embodiments, the biosynthesized phytohormones disposed in the aqueous fraction may comprise 60-70%. In some embodiments, the biosynthesized phytohormones disposed in the aqueous fraction may comprise 70-80%. In some embodiments, the biosynthesized phytohormones disposed in the aqueous fraction may comprise at least 30%. In some embodiments, the biosynthesized phytohormones disposed in the aqueous fraction may comprise at least 40%. In some embodiments, the biosynthesized phytohormones disposed in the aqueous fraction may comprise at least 50%. In some embodiments, the biosynthesized phytohormones disposed in the aqueous fraction may comprise at least 60%. In some embodiments, the biosynthesized phytohormones disposed in the aqueous fraction may comprise at least 70%. In some embodiments, the biosynthesized phytohormones disposed in the aqueous fraction may comprise at least 80%.

In some embodiments, the phytohormone enriched microalgae may be pasteurized without substantial degradation of the phytohormone content. In some embodiments, the phytohormone content of the phytohormone enriched microalgae may decrease by less than 30% after being subjected to a pasteurization process. In some embodiments, the phytohormone content of the phytohormone enriched microalgae may decrease by less than 25% after being subjected to a pasteurization process. In some embodiments, the phytohormone content of the phytohormone enriched microalgae may decrease by less than 20% after being subjected to a pasteurization process. In some embodiments, the phytohormone content of the phytohormone enriched microalgae may decrease by less than 15% after being subjected to a pasteurization process. In some embodiments, the phytohormone content of the phytohormone enriched microalgae may decrease by less than 10% after being subjected to a pasteurization process. In some embodiments, the phytohormone content of the phytohormone enriched microalgae may decrease by less than 5% after being subjected to a pasteurization process. In some embodiments, the phytohormone content of the phytohormone enriched microalgae may decrease by less than 1% after being subjected to a pasteurization process.

In one aspect, many plants may benefit from the application of a liquid composition that provides a bio-stimulatory effect. Non-limiting examples of plant families that can benefit from such composition application can comprise plants from the following: Solanaceae, Fabaceae (Leguminosae), Poaceae, Rosaceae, Vitaceae, Brassicaeae (Cruciferae), Caricaceae, Malvaceae, Sapindaceae, Anacardiaceae, Rutaceae, Moraceae, Convolvulaceae, Lamiaceae, Verbenaceae, Pedaliaceae, Asteraceae (Compositae), Apiaceae (Umbelliferae), Araliaceae, Oleaceae, Ericaceae, Actinidaceae, Cactaceae, Chenopodiaceae, Polygonaceae, Theaceae, Lecythidaceae, Rubiaceae, Papveraceae, Illiciaceae Grossulariaceae, Myrtaceae, Juglandaceae, Bertulaceae, Cucurbitaceae, Asparagaceae (Liliaceae), Alliaceae (Liliceae), Bromeliaceae, Zingieraceae, Muscaceae, Areaceae, Dioscoreaceae, Myristicaceae, Annonaceae, Euphorbiaceae, Lauraceae, Piperaceae, and Proteaceae.

The Solanaceae plant family includes a large number of agricultural crops, medicinal plants, spices, and ornamentals in its over 2,500 species. Taxonomically classified in the Plantae kingdom, Tracheobionta (subkingdom), Spermatophyta (superdivision), Magnoliophyta (division), Magnoliopsida (class), Asteridae (subclass), and Solanales (order), the Solanaceae family includes, but is not limited to, potatoes, tomatoes, eggplants, various peppers, tobacco, and petunias. Plants in the Solanaceae can be found on all the continents, excluding Antarctica, and thus have a widespread importance in agriculture across the globe.

The Fabaceae plant family (also known as the Leguminosae) comprises the third largest plant family with over 18,000 species, including a number of important agricultural and food plants. Taxonomically classified in the Plantae kingdom, Tracheobionta (subkingdom), Spermatophyta (superdivision), Magnoliophyta (division), Manoliopsida (class), Rosidae (subclass), and Fabales (order), the Fabaceae family includes, but is not limited to, soybeans, beans, green beans, peas, chickpeas, alfalfa, peanuts, sweet peas, carob, and liquorice. Plants in the Fabaceae family can range in size and type, including but not limited to, trees, small annual herbs, shrubs, and vines, and typically develop legumes. Plants in the Fabaceae family can be found on all the continents, excluding Antarctica, and thus have a widespread importance in agriculture across the globe. Besides food, plants in the Fabaceae family can be used to produce natural gums, dyes, and ornamentals.

The Poaceae plant family supplies food, building materials, and feedstock for fuel processing. Taxonomically classified in the Plantae kingdom, Tracheobionta (subkingdom), Spermatophyta (superdivision), Magnoliophyta (division), Liliopsida (class), Commelinidae (subclass), and Cyperales (order), the Poaceae family includes, but is not limited to, flowering plants, grasses, and cereal crops such as barely, corn, lemongrass, millet, oat, rye, rice, wheat, sugarcane, and sorghum. Types of turf grass that can be found in Arizona (e.g., and other locations) include, but are not limited to, hybrid Bermuda grasses (e.g., tifgreen 328, tifway 419, tifsport).

The Rosaceae plant family includes flowering plants, herbs, shrubs, and trees. Taxonomically classified in the Plantae kingdom, Tracheobionta (subkingdom), Spermatophyta (superdivision), Magnoliophyta (division), Magnoliopsida (class), Rosidae (subclass), and Rosales (order), the Rosaceae family includes, but is not limited to, almond, apple, apricot, blackberry, cherry, nectarine, peach, plum, raspberry, strawberry, and quince.

The Vitaceae plant family includes flowering plants and vines. Taxonomically classified in the Plantae kingdom, Tracheobionta (subkingdom), Spermatophyta (superdivision), Magnoliophyta (division), Magnoliopsida (class), Rosidae (subclass), and Rhammales (order), the Vitaceae family includes, but is not limited to, grapes.

In the production of fruit from plants, particular attention is often paid to the beginning stage of growth, where the plant emerges and matures into establishment. In this aspect, treating a seed, seedling, or plant to directly improve the germination, emergence, and maturation of the plant, or to indirectly enhance the microbial soil community surrounding the seed or seedling, can be beneficial when starting the plant on the path to marketable production. A typical standard used to assess plant emergence is achievement of the hypocotyl stage, where a stem is visibly protruding from the soil. A typical standard used for assessing maturation of the plant is the achievement of the cotyledon stage, where two leaves are visibly formed on the emerged stem. In this aspect, another important assessment of the plant is the production of fruit from plants, including the yield and quality of fruit; which may be quantified as the number, weight, color, firmness, ripeness, moisture, degree of insect infestation, degree of disease or rot, and/or a degree of sunburn of the fruit.

In this aspect, in one implementation, a method may be devised to treat a plant to directly improve the characteristics of the plant, to indirectly enhance the chlorophyll level of the plant for photosynthetic capabilities, and/or to enhance the health of the plant's leaves, roots, and shoots. For example, this type of treatment may enable robust production of fruit, thereby increasing the efficiency of marketable production. As an example, marketable and unmarketable designations may apply to both the plant and resulting fruit, and may be defined differently based on the end use of the product. For example, the fruit and/or plant may be designated as fresh market produce, and/or may be processed for inclusion as an ingredient in a composition (e.g., or stand-alone product). A marketable determination may assess plant/fruit qualities, such as, but not limited to, color, insect damage, blossom end rot, softness, and/or sunburn, amongst other things. The term total production may incorporate both marketable and unmarketable plants and fruit. The ratio of marketable plants or fruit to unmarketable plants or fruit may be referred to as utilization and expressed as a percentage. The utilization may be used as an indicator of the efficiency of the agricultural process, as it can be indicative of the success of a production of marketable plants or fruit. For example, higher efficiency may obtain a higher financial return for the grower, whereas total production may not provide such an indication of success.

In this aspect, a method has been devised, as described herein, that can help achieve improvements in emergence, maturation, and/or yield of plants, amongst other things. In one implementation, in this aspect, targeted seeds, plants, and/or soil can be treated with an appropriate concentration (e.g., determined and targeted based at least on the target plant, conditions, and expected results) of IAA enriched microalgae based composition, in a dried and/or liquid solution form. In one implementation, microalgae may be cultured in heterotrophic, mixotrophic, and/or phototrophic conditions, and combined with one or more IAA precursors, as previously described. For example, culturing microalgae in heterotrophic conditions can comprise supplying organic carbon (e.g., acetic acid, acetate, glucose, etc.) to cells in an aqueous culture medium comprising trace metals and nutrients (e.g., nitrogen, phosphorus). As another example, culturing microalgae in mixotrophic conditions can comprise supplying light and organic carbon (e.g., acetic acid, acetate, glucose, etc.) to cells in an aqueous culture medium comprising trace metals and nutrients (e.g., nitrogen, phosphorus). As another example, culturing microalgae in phototrophic conditions can comprise supplying light and inorganic carbon (e.g., carbon dioxide) to cells in an aqueous culture medium comprising trace metals and nutrients (e.g., nitrogen, phosphorus).

In some embodiments, resulting IAA enriched microalgae cells may be harvested from a culture and used as whole cells in a liquid composition for application to seeds and plants. In other embodiments the harvested resulting IAA enriched microalgae cells may be subjected to downstream processing, and the resulting biomass or extract may be used in a dried composition (e.g., powder, pellet) or a liquid composition (e.g., suspension, solution) for application to plants, plant parts, soil, or a combination thereof. Non-limiting examples of downstream processing can comprise: drying the cells, lysing the cells, and/or subjecting the harvested cells to a solvent or supercritical carbon dioxide extraction process to isolate a target oil, protein, or other desired product. In some embodiments, the extracted (i.e., residual) biomass remaining from an extraction process may be used alone or in combination with other microalgae, or microalgae extracts, in a liquid composition for application to plants, plant parts, soil, or a combination thereof. As one example, by subjecting the resulting IAA enriched microalgae to an extraction process, the resulting biomass can be transformed from a natural whole state to a lysed condition, where the cell is missing a significant amount of the natural components, thus differentiating the extracted microalgae biomass from that which is found in nature. As an example, excreted products from microalgae may also be isolated from a microalgae culture by applying downstream processing methods.

In some embodiments, the resulting IAA enriched microalgae may be the dominant active ingredient source in the composition. In some embodiments, the IAA enriched microalgae population of the composition may comprise whole biomass, substantially extracted biomass, excreted products (e.g., excreted polysaccharides [EPS]), extracted protein, or extracted oil. In some embodiments, IAA enriched microalgae can comprise at least 99% of the active ingredient sources of the composition. In some embodiments, IAA enriched microalgae can comprise at least 95% of the active ingredient sources of the composition. In some embodiments, IAA enriched microalgae can comprise at least 90% of the active ingredient sources of the composition. In some embodiments, IAA enriched microalgae can comprise at least 80% of the active ingredient sources of the composition. In some embodiments, IAA enriched microalgae can comprise at least 70% of the active ingredient sources of the composition. In some embodiments, IAA enriched microalgae can comprise at least 60% of the active ingredient sources of the composition. In some embodiments, IAA enriched microalgae can comprise at least 50% of the active ingredient sources of the composition. In some embodiments, IAA enriched microalgae can comprise at least 40% of the active ingredient sources of the composition. In some embodiments, IAA enriched microalgae can comprise at least 30% of the active ingredient sources of the composition. In some embodiments, IAA enriched microalgae can comprise at least 20% of the active ingredient sources of the composition. In some embodiments, IAA enriched microalgae can comprise at least 10% of the active ingredient sources of the composition. In some embodiments, IAA enriched microalgae can comprise at least 5% of the active ingredient sources of the composition. In some embodiments, IAA enriched microalgae can comprise at least 1% of the active ingredient sources of the composition. In some embodiments, the composition may lack any detectable amount of any other active ingredient source other than IAA enriched microalgae.

In some embodiments, IAA enriched microalgae biomass, excreted products, or extracts may also be mixed with biomass or extracts from other plants, microalgae, macroalgae, seaweeds, and kelp. In some embodiments, IAA enriched microalgae biomass, excreted products, or extracts may also be mixed with fish oil. Non-limiting examples of other plants, macroalgae, seaweeds, and kelp fractions that may be combined with microalgae cells may comprise species of Lemna, Gracilaria, Kappaphycus, Ascophyllum, Macrocystis, Fucus, Laminaria, Sargassum, Turbinaria, and Durvilea. In further embodiments, the extracts may comprise, but are not limited to, liquid extract from a species of Kappaphycus. In some embodiments, the extracts may comprise 50% or less by volume of the composition. In some embodiments, the extracts may comprise 40% or less by volume of the composition. In some embodiments, the extracts may comprise 30% or less by volume of the composition. In some embodiments, the extracts may comprise 20% or less by volume of the composition. In some embodiments, the extracts may comprise 10% or less by volume of the composition. In some embodiments, the extracts may comprise 5% or less by volume of the composition. In some embodiments, the extracts may comprise 4% or less by volume of the composition. In some embodiments, the extracts may comprise 3% or less by volume of the composition. In some embodiments, the extracts may comprise 2% or less by volume of the composition. In some embodiments, the extracts may comprise 1% or less by volume of the composition.

In some embodiments, the IAA enriched microalgae may be previously frozen and thawed before inclusion in the liquid composition. In some embodiments, the IAA enriched microalgae may not have been subjected to a previous freezing or thawing process. In some embodiments, the IAA enriched microalgae whole cells have not been subjected to a drying process. The cell walls of the IAA enriched microalgae of the composition have not been lysed or disrupted, and the microalgae cells have not been subjected to an extraction process or process that pulverizes the cells. The IAA enriched microalgae whole cells are not subjected to a purification process for isolating the microalgae whole cells from the accompanying constituents of the culturing process (e.g., trace nutrients, residual organic carbon, bacteria, cell debris, cell excretions), and thus the whole output from the IAA enriched microalgae culturing process comprising whole microalgae cells, culture medium, cell excretions, cell debris, bacteria, residual organic carbon, and trace nutrients, is used in the liquid composition for application to plants. In some embodiments, the IAA enriched microalgae whole cells and the accompanying constituents of the culturing process are concentrated in the composition. In some embodiments, the IAA enriched microalgae whole cells and the accompanying constituents of the culturing process are diluted in the composition to a low concentration. The IAA enriched microalgae whole cells of the composition are not fossilized. In some embodiments, the IAA enriched microalgae whole cells are not maintained in a viable state in the composition for continued growth after the method of using the composition in a soil or foliar application. In some embodiments, the IAA enriched microalgae based composition may be biologically inactive after the composition is prepared. In some embodiments, the IAA enriched microalgae based composition may be substantially biologically inactive after the composition is prepared. In some embodiments, the IAA enriched microalgae based composition may increase in biological activity after the prepared composition is exposed to air.

In some embodiments, a liquid composition may comprise low concentrations of bacteria contributing to the solids percentage of the composition in addition to the IAA enriched microalgae cells. Examples of bacteria found in non-axenic mixotrophic conditions may be found in WO2014/074769A2 (Ganuza, et al.), hereby incorporated by reference. A live bacteria count may be determined using methods known in the art such as plate counts, plate counts using Petrifilm available from 3M (St. Paul, Minn.), spectrophotometric (turbidimetric) measurements, visual comparison of turbidity with a known standard, direct cell counts under a microscope, cell mass determination, and measurement of cellular activity. Live bacteria counts in a non-axenic mixotrophic microalgae culture may range from 10⁴ to 10⁹ CFU/mL, and may depend on contamination control measures taken during the culturing of the microalgae. The level of bacteria in the composition may be determined by an aerobic plate count which quantifies aerobic colony forming units (CFU) in a designated volume. In some embodiments, the composition comprises an aerobic plate count of 40,000-400,000 CFU/mL. In some embodiments, the composition comprises an aerobic plate count of 40,000-100,000 CFU/mL. In some embodiments, the composition comprises an aerobic plate count of 100,000-200,000 CFU/mL. In some embodiments, the composition comprises an aerobic plate count of 200,000-300,000 CFU/mL. In some embodiments, the composition comprises an aerobic plate count of 300,000-400,000 CFU/mL.

In some embodiments, the IAA enriched microalgae based composition can be supplemented with a supplemental nutrient such as nitrogen, phosphorus, or potassium to increase the levels of the added supplement within the composition to at least 1% of the total composition (i.e., addition of N, P, or K to increase levels at least 1-0-0, 0-1-0, 0-0-1, or combinations thereof). In some embodiments, the IAA enriched microalgae composition may be supplemented with nutrients such as, but not limited to, calcium, magnesium, silicon, sulfur, iron, manganese, zinc, copper, boron, molybdenum, chlorine, sodium, aluminum, vanadium, nickel, cerium, dysprosium, erbium, europium, gadolinium, holmium, lanthanum, lutetium, neodymium, praseodymium, promethium, samarium, scandium, terbium, thulium, ytterbium, and yttrium. In some embodiments, the supplemented nutrient is not taken up, chelated, or absorbed by the IAA enriched microalgae. In some embodiments, the concentration of the supplemental nutrient may comprise 1-50 g per 100 g of the composition.

In some embodiments, a liquid composition comprising IAA enriched microalgae may be stabilized by heating and cooling in a pasteurization process. As shown in the Examples discussed below, the effectiveness of the active ingredients of the IAA enriched microalgae based composition can be maintained in at least one characteristic of a plant after being subjected to the heating and cooling of a pasteurization process. In other embodiments, liquid compositions with whole cells or processed cells (e.g., dried, lysed, extracted) of IAA enriched microalgae cells may not need to be stabilized by pasteurization. For example, IAA enriched microalgae cells that have been processed, such as by drying, lysing, and extraction, or extracts may comprise such low levels of bacteria that a liquid composition may remain stable without being subjected to the heating and cooling of a pasteurization process.

In some embodiments, the composition may be heated to a temperature in the range of 50-70° C. In some embodiments, the composition may be heated to a temperature in the range of 55-65° C. In some embodiments, the composition may be heated to a temperature in the range of 58-62° C. In some embodiments, the composition may be heated to a temperature in the range of 50-60° C. In some embodiments, the composition may be heated to a temperature in the range of 60-70° C.

In some embodiments, the composition may be heated for a time period in the range of 90-150 minutes. In some embodiments, the composition may be heated for a time period in the range of 110-130 minutes. In some embodiments, the composition may be heated for a time period in the range of 90-100 minutes. In some embodiments, the composition may be heated for a time period in the range of 100-110 minutes. In some embodiments, the composition may be heated for a time period in the range of 110-120 minutes. In some embodiments, the composition may be heated for a time period in the range of 120-130 minutes. In some embodiments, the composition may be heated for a time period in the range of 130-140 minutes. In some embodiments, the composition may be heated for a time period in the range of 140-150 minutes.

After the step of heating or subjecting the liquid composition to high temperatures is complete, the compositions may be cooled at any rate to a temperature that is safe to work with. In one non-limiting embodiment, the composition may be cooled to a temperature in the range of 35-45° C. In some embodiments, the composition may be cooled to a temperature in the range of 36-44° C. In some embodiments, the composition may be cooled to a temperature in the range of 37-43° C. In some embodiments, the composition may be cooled to a temperature in the range of 38-42° C. In some embodiments, the composition may be cooled to a temperature in the range of 39-41° C. In further embodiments, the pasteurization process may be part of a continuous production process that also involves packaging, and thus the liquid composition may be packaged (e.g., bottled) directly after the heating or high temperature stage without a cooling step.

In some embodiments, the composition may comprise 5-30% solids by weight of microalgae cells (i.e., 5-30 g of microalgae cells/100 mL of the liquid composition). In some embodiments, the composition may comprise 5-20% solids by weight of microalgae cells. In some embodiments, the composition may comprise 5-15% solids by weight of microalgae cells. In some embodiments, the composition may comprise 5-10% solids by weight of microalgae cells. In some embodiments, the composition may comprise 10-20% solids by weight of microalgae cells. In some embodiments, the composition may comprise 10-20% solids by weight of microalgae cells. In some embodiments, the composition may comprise 20-30% solids by weight of microalgae cells. In some embodiments, further dilution of the microalgae cells percent solids by weight may be occur before application for low concentration applications of the composition.

In some embodiments, the composition may comprise less than 1% by weight of IAA enriched microalgae biomass or extracts (i.e., less than 1 g of microalgae derived product/100 mL of the liquid composition). In some embodiments, the composition may comprise less than 0.9% by weight of IAA enriched microalgae biomass or extracts. In some embodiments, the composition may comprise less than 0.8% by weight of IAA enriched microalgae biomass or extracts. In some embodiments, the composition may comprise less than 0.7% by weight of IAA enriched microalgae biomass or extracts. In some embodiments, the composition may comprise less than 0.6% by weight of IAA enriched microalgae biomass or extracts. In some embodiments, the composition may comprise less than 0.5% by weight of IAA enriched microalgae biomass or extracts. In some embodiments, the composition may comprise less than 0.4% by weight of IAA enriched microalgae biomass or extracts. In some embodiments, the composition may comprise less than 0.3% by weight of microalgae biomass or extracts. In some embodiments, the composition may comprise less than 0.2% by weight of IAA enriched microalgae biomass or extracts. In some embodiments, the composition may comprise less than 0.1% by weight of IAA enriched microalgae biomass or extracts. In some embodiments, the composition may comprise at least 0.0001% by weight of IAA enriched microalgae biomass or extracts. In some embodiments, the composition may comprise at least 0.001% by weight of IAA enriched microalgae biomass or extracts. In some embodiments, the composition may comprise at least 0.01% by weight of IAA enriched microalgae biomass or extracts. In some embodiments, the composition may comprise at least 0.1% by weight of IAA enriched microalgae biomass or extracts. In some embodiments, the composition may comprise 0.0001-1% by weight of microalgae biomass or extracts. In some embodiments, the composition may comprise 0.0001-0.001% by weight of IAA enriched microalgae biomass or extracts. In some embodiments, the composition may comprise 0.001-.01% by weight of IAA enriched microalgae biomass or extracts. In some embodiments, the composition may comprise 0.01-0.1% by weight of IAA enriched microalgae biomass or extracts. In some embodiments, the composition may comprise 0.1-1% by weight of IAA enriched microalgae biomass or extracts.

In some embodiments, an application concentration of 0.1% of IAA enriched microalgae biomass or extract equates to 0.04 g of IAA enriched microalgae biomass or extract in 40 mL of a composition. While the desired application concentration to a plant may be 0.1% of IAA enriched microalgae biomass or extract, the composition may be packaged as a 10% concentration (0.4 g in 40 mL of a composition). Thus, a desired application concentration of 0.1% would utilize 6,000 mL of the 10% IAA enriched microalgae biomass or extract in the 100 gallons of water applied to the assumption of 15,000 plants in an acre, which is equivalent to an application rate of about 1.585 gallons per acre. In some embodiments, a desired application concentration of 0.01% of IAA enriched microalgae biomass or extract using a 10% concentration composition equates to an application rate of about 0.159 gallons per acre. In some embodiments, a desired application concentration of 0.001% of IAA enriched microalgae biomass or extract using a 10% concentration composition equates to an application rate of about 0.016 gallons per acre. In some embodiments, a desired application concentration of 0.0001% of IAA enriched microalgae biomass or extract using a 10% concentration composition equates to an application rate of about 0.002 gallons per acre.

In another non-limiting embodiment, correlating the application of the IAA enriched microalgae biomass or extract on a per plant basis using the assumption of 15,000 plants per acre, the composition application rate of 1 gallon per acre is equal to about 0.25 mL per plant =0.025 g per plant =25 mg of microalgae biomass or extract per plant. The water requirement assumption of 100 gallons per acre is equal to about 35 mL of water per plant. Therefore, 0.025 g of IAA enriched microalgae biomass or extract in 35 mL of water is equal to about 0.071 g of IAA enriched microalgae biomass or extract per 100 mL of composition equates to about a 0.07% application concentration. In some embodiments, the IAA enriched microalgae biomass or extract based composition may be applied at a rate in a range as low as about 0.001-10 gallons per acre, or as high as up to 150 gallons per acre.

In some embodiments, stabilizing components can be added to a resulting product, which can aid in stabilizing the composition to mitigate proliferation of unwanted microorganisms (e.g., yeast, mold) and prolong shelf life. As an example, these stabilizing component may not be used for the improvement of plant germination, emergence, maturation, quality, and yield, but instead product stabilization. As an example, such inactive but stabilizing components may comprise an acid, such as, but not limited to, phosphoric acid or citric acid; and/or a yeast and mold inhibitor, such as, but not limited to potassium sorbate. In some embodiments, the stabilizing components can be suitable for use with plants, and may not inhibit the growth or health of the plant. Alternatively, the stabilizing components can contribute to nutritional properties of the liquid composition, such as, but not limited to, the levels of nitrogen, phosphorus, or potassium.

In some embodiments, the composition may comprise less than 0.3% phosphoric acid. In some embodiments, the composition may comprise 0.01-0.3% phosphoric acid. In some embodiments, the composition may comprise 0.05-0.25% phosphoric acid. In some embodiments, the composition may comprise 0.01-0.1% phosphoric acid. In some embodiments, the composition may comprise 0.1-0.2% phosphoric acid. In some embodiments, the composition may comprise 0.2-0.3% phosphoric acid. In some embodiments, the composition may comprise less than 0.3% citric acid. In some embodiments, the composition may comprise 0.01-0.3% citric acid. In some embodiments, the composition may comprise 0.05-0.25% citric acid. In some embodiments, the composition may comprise 0.01-0.1% citric acid. In some embodiments, the composition may comprise 0.1-0.2% citric acid. In some embodiments, the composition may comprise 0.2-0.3% citric acid.

In some embodiments, the composition may comprise less than 0.5% potassium sorbate. In some embodiments, the composition may comprise 0.01-0.5% potassium sorbate. In some embodiments, the composition may comprise 0.05-0.4% potassium sorbate. In some embodiments, the composition may comprise 0.01-0.1% potassium sorbate. In some embodiments, the composition may comprise 0.1-0.2% potassium sorbate. In some embodiments, the composition may comprise 0.2-0.3% potassium sorbate. In some embodiments, the composition may comprise 0.3-0.4% potassium sorbate. In some embodiments, the composition may comprise 0.4-0.5% potassium sorbate.

In some embodiments, the composition can be in a liquid form that is substantially comprised of water. In some embodiments, the composition may comprise 70-99% water. In some embodiments, the composition may comprise 85-95% water. In some embodiments, the composition may comprise 70-75% water. In some embodiments, the composition may comprise 75-80% water. In some embodiments, the composition may comprise 80-85% water. In some embodiments, the composition may comprise 85-90% water. In some embodiments, the composition may comprise 90-95% water. In some embodiments, the composition may comprise 95-99% water. In these embodiments, the liquid nature and high water content of the composition can facilitate administration of the composition in a variety of manners, such as, but not limit to: flowing through an irrigation system, flowing through an above ground drip irrigation system, flowing through a buried drip irrigation system, flowing through a central pivot irrigation system, sprayers, sprinklers, water cans and other fluid application techniques.

In some embodiments, a liquid composition may be used substantially immediately after formulation, or may be stored in one or more containers for later use. In some embodiments, the composition may be stored out of direct sunlight. In some embodiments, the composition may be refrigerated. In some embodiments, the composition may be stored at 1-10° C. In some embodiments, the composition may be stored at 1-3° C. In some embodiments, the composition may be stored at 3-5° C. In some embodiments, the composition may be stored at 5-8° C. In some embodiments, the composition may be stored at 8-10° C.

In some embodiments, the liquid composition may be administered to a seed or plant in an amount effective to produce an enhanced characteristic in a target plant, when compared to a substantially identical population of untreated seeds or plants. Such enhanced characteristics may comprise: accelerated seed germination, accelerated seedling emergence, improved seedling emergence, improved leaf formation, accelerated leaf formation, improved plant maturation, accelerated plant maturation, increased plant yield, increased plant growth, increased plant quality, increased plant health, increased fruit yield, increased fruit growth, and/or increased fruit quality. Non-limiting examples of such enhanced characteristics may comprise: accelerated achievement of the hypocotyl stage, accelerated protrusion of a stem from the soil, accelerated achievement of the cotyledon stage, accelerated leaf formation, increased marketable plant weight, increased marketable plant yield, increased marketable fruit weight, increased production plant weight, increased production fruit weight, increased utilization (indicator of efficiency in the agricultural process based on ratio of marketable fruit to unmarketable fruit), increased chlorophyll content (indicator of plant health), increased plant weight (indicator of plant health), increased root weight (indicator of plant health), increased shoot weight (indicator of plant health), increased plant height, increased thatch height, increased resistance to salt stress, increased plant resistance to heat stress (temperature stress), increased plant resistance to heavy metal stress, increased plant resistance to drought, increased plant resistance to disease, improved color, reduced insect damage, reduced blossom end rot, and/or reduced sun burn. Such enhanced characteristics may occur individually in a plant, or in combinations of multiple enhanced characteristics.

In some embodiments, after harvest of the resulting IAA enriched microalgae from the culturing vessel, the microalgae biomass may be dried or dehydrated to form a composition of dried microalgae biomass (i.e., reduced moisture content). The microalgae biomass may be dried by at least one method selected from the group consisting of: freeze drying (or lypohilization), drum (or rotary) drying, spray drying, crossflow air drying, solar drying, vacuum shelf drying, pulse combustion drying, flash drying, furnace drying, belt conveyor drying, and refractance window drying. In some embodiments, the microalgae cells may be dried by a combination of two or more methods, such as in a process with multiple drying methods in series. The process of drying the microalgae biomass may reduce the percent moisture (on a wet basis) to the range of about 1-15% and result in a cake, flakes, or a powder, which is more uniform and more stable than the wet culture of microalgae. In some embodiments, the dried microalgae cells may be intact. In some embodiments, the dried microalgae cells may be lysed or disrupted. In some embodiments, the microalgae cells may be lysed or disrupted prior to or after drying by mechanical, electrical, acoustic, or chemical means. In some embodiments, drying the microalgae cells achieves an acceptable product stability for storage, with the reduction or elimination of chemical stabilizers. The composition may be stored in any suitable container such as, but not limited to, a bag, bucket, jug, tote, or bottle.

In some embodiments, the dried IAA enriched microalgae biomass may have a moisture content of 1-15% on a wet basis. In some embodiments, the dried IAA enriched microalgae biomass may have a moisture content of 1-2% on a wet basis. In some embodiments, the dried IAA enriched microalgae biomass may have a moisture content of 2-3% on a wet basis. In some embodiments, the dried IAA enriched microalgae biomass may have a moisture content of 3-5% on a wet basis. In some embodiments, the dried IAA enriched microalgae biomass may have a moisture content of 5-7% on a wet basis. In some embodiments, the dried IAA enriched microalgae biomass may have a moisture content of 7-10% on a wet basis. In some embodiments, the dried IAA enriched microalgae biomass may have a moisture content of 10-12% on a wet basis. In some embodiments, the dried IAA enriched microalgae biomass may have a moisture content of 12-15% on a wet basis. In some embodiments, the dried IAA enriched microalgae biomass may have a moisture content of 1-8% on a wet basis. In some embodiments, the dried IAA enriched microalgae biomass may have a moisture content of 8-15% on a wet basis.

The various drying processes may have different capabilities such as, but not limited to, the amount of moisture that may be removed, the preservation of metabolites (e.g., proteins, lipids, pigments, carbohydrates, polysaccharides, soluble nitrogen, phytohormones), and the effect on the cell wall or membrane. For example, loss of protein in Spirulina biomass has been found to increase proportionally as the drying temperature increases. Additionally, drying at high temperatures has been shown to alter polymer chains, alter interactions between polysaccharide and glycoprotein, and increase bound water content of polysaccharides. Pigments and fatty acids are also known to oxidize and de-stabilize to different degrees in different drying processes. The effectiveness of each drying method may also vary based on the microalgae species due to different physical characteristics of the microalgae (e.g., sheer sensitivity, cell size, cell wall thickness and composition). The method of drying and drying method parameters may also result in a structural change to the microalgae cell such as, but not limited to, increased porosity in the cell wall, changes in the cell wall make up or bonds, and measurable changes in cell characteristics (e.g., elasticity, viscosity, digestibility); as wells as functional differences when applied to plants that can be measured in changes in plant performance or plant characteristics. Drying microalgae with a combination of methods in series may also result in structural and functional changes, minimize structural and functional changes, or increase the effectiveness for a particular type of microalgae.

Drum drying comprises the use of sloped, rotating cylinders which use gravity to move the microalgal biomass from one end to the other. Drum drying may be conducted with direct contact between a hot gas and the microalgal biomass, or indirect heating in which the gas and microalgal biomass is separated by a barrier such as a steel shell. A non-limiting example of a drum drying process for Scenedesmus may comprise 10 seconds of heating at 120° C. Possible effects to the microalga biomass in a drum drying process include sterilization of the biomass, and breaking of the cell wall. Microalgal biomass that is drum dried may have higher digestibility than microalgal biomass that is spray dried.

Freeze drying comprises freezing the microalgal biomass and then transferring the frozen biomass to a vacuum chamber with reduced pressure (e.g., 4.6 Ton). The ice in the microalgal biomass changes to vapor through sublimation which is collected on an extremely cold condenser and removed from the vacuum chamber. Freeze drying typically minimizes the degradation of unsaturated fatty acids and pigments (e.g., carotenoids) through oxidation, which preserves the nutritional value of the microalgal biomass. Although the targeted removal of water in the freeze drying process is beneficial, the process is very costly and time consuming which makes freeze drying impractical for many commercial applications. In some embodiments, microalgae dried by freeze drying may comprise 2-6% moisture (on a wet basis). A non-limiting example of a freeze drying process for Scenedesmus may comprise 24 hours at -84° C. Freeze drying is known to maintain the integrity of the microalgal cell, but is also known been known in some cases to disrupt the cell or increase the pore size in the cell wall. In Scenedesmus, freeze drying was found to decrease rigidity, increase surface area by 165%, and increase pore size by 19% of the cells (see eSEM images below). In Phaeodactylum ricornutum, freeze drying had no noticeable effect on the total lipid content, made the cells more susceptible to lipolysis (i.e., breakdown of lipids, hydrolysis of triglycerides into glycerol and free fatty acids) upon storage than spray dried cells, and made the cells less susceptible to oxidation than spray dried cells.

Spray drying comprises atomizing an aqueous microalgae culture into droplets sprayed downwardly in a vertical tower through which hot gases pass downward. The gas stream may be exhausted through a cyclonic separator. The process of spray drying is expensive, but slightly cheaper than freeze drying. Spray drying has become the method of choice for high value products (e.g., >$1,000 /ton). With an appropriate type of burner, oxygen can be mostly eliminated from the recycled drying gas, which prevents the oxidation of oxygen sensitive products (e.g., carotenoids). In some embodiments, microalgae dried by spray drying may comprise 1-7% moisture (on a wet basis). Examples of spray drying systems include: box dryers, tall-form spray dryers, fluidized bed dryers, and moving fluidized bed dryers (e.g., FilterMat spray dryer GEA Process Engineering Inc.). An open cycle spray dryer with a particular direct fired air heater may operate at elevated temperatures (e.g., 60-93° C.) and high oxygen concentrations (e.g., 19-20%). The possible effects of spray drying on microalgal biomass include rupturing the cells walls, reduction of protein content by 10-15%, significant deterioration of pigments (depending on the oxygen concentration), and a lower digestibility than drum drying. In Phaeodactylum ricornutum, spray drying had no noticeable effect on the total lipid content, made the cells less susceptible to lipolysis than freeze drying, and made the cells more susceptible to oxidation than freeze drying (possibly due to the breakdown of protective carotenoids).

Crossflow air drying uses movement of heated air across a layer of microalgae on a tray, which is a modification of indirect solar and convection oven driers. Crossflow air drying is faster than solar drying, cheaper than drum drying, and is known to typically not break the microalgal cell wall. In some embodiments, microalgae dried by crossflow air drying may comprise 8-12% moisture (on a wet basis). Non-limiting examples of crossflow air drying for Spirulina may comprise: 1) a temperature of 62° C. for 14 hours, 2) a temperature of 50-60° C., a relative humidity of 7-10%, an air velocity of 1.5 m/s, and a duration of 150-220 minutes, 3) a temperature of 40-60° C. and an air velocity of 1.9-3.8 m/s, and 4) temperatures of 50-70° C. for layers of 3-7 mm in a perforated tray with parallel air flow. Crossflow air drying of Spirulina has shown a loss in protein of about 17% and a loss in phycocyanin of 37-50%. Particularly, degradation of phycocyanin was found to occur above 60° C., but there was no significant change in the fatty acid composition in the crossflow air drying methods.

Non-limiting examples of crossflow air drying of Chlorella kessleri and Chlamydomonas reinhardtii may comprise a temperature of 55° C. for more than 5 hours. Crossflow air drying of Chlorella kessleri and Chlamydomonas reinhardtii has produced a reduction of chlorophyll relative to the dry cell weight, an increase of total fatty acid content relative to the dry cell, a decrease of polar lipids relative to the dry cell weight, and a decrease in the availability of nutritional salts (e.g., S, N). A cell's sensitivity to air drying stress (as measured through the change in chlorophyll) may be correlated to the properties of the cell wall. For example, the crossflow air dried Chlamydomonas reinhardtii (hydroxyproline-rich glucoprotein based cell walls) had a larger decrease in chlorophyll than the Chlorella kessleri (sugar based cell walls), which may be associated with the cell wall's ability to restructure in S and N deficient conditions. In a non-limiting example of drying 5-7 mm thick layers of Aphanothece microscopia Nageli at temperatures of 40-60° C. with parallel air flow of 1.5 m/s, it was found that drying conditions influenced the concentrations of protein, carbohydrates, and lipids in the biomass.

Solar drying methods may comprise the use of direct solar radiation to dry microalgae on sand or a plastic sheet, or the indirect use of solar radiation to heat air that is circulated around microalgae in a dryer. Direct solar drying is strongly weather dependent, slow, and may require a short duration of high heat (e.g., 120° C.) to increase the biological value of the microalgal biomass. A non-limiting example of a direct solar drying process for Scenedesmus may comprise a 1,500 micron thickness white plastic drying bed liner, a temperature of 25-30° C., and a duration of 72 hours. The possible effects of direct solar drying on microalgal biomass include chlorophyll degradation, overheating of the biomass, and creation of an unpleasant odor. Indirect solar drying prevents overheating, has a higher drying rate than direct solar drying, but produces a less attractive profile in the final product. An indirect solar drying method for microalgae may comprise temperature of 65-70° C. for 0.5-6 hours.

Drying of a thin film of microalgal biomass in a convection oven is a fairly common practice performed in scientific literature to test the biomass going through further processing, but may be less practical for many commercial applications. Thin film convection oven drying has been demonstrated in the literature with species of Chlorella, Chlamydomonas, and Scenedesmus. In some embodiments, microalgae dried by oven drying may comprise 6-10% moisture (on a wet basis). Thin film convection oven drying methods may comprise temperatures of 30-90° C., and durations of 4-12 hours. Thin film convection oven dried microalgal biomass showed no significant change in the fatty acid profile and a slight decrease in the degree of unsaturation of fatty acids at higher temperature for ruptured cells (likely due to oxidation causing cleavage of unsaturated bonds).

Microalgae may be dried in thin layers with heat at a reduced pressure. Non-limiting examples of drying of Spirulina in layers within a vacuum may comprise temperatures of 50-65° C. and a pressure of 0.05-0.06 atm. Possible effects on the microalgae that may result from vacuum shelf drying include development of a hygroscopic property (i.e., ability to attract and hold water particles from the surrounding environment by absorption or adsorption) and development of a porous structure.

Pulse combustion drying uses a blast of controlled heat to flash dry the microalgae. Air is pumped into a combustion chamber, mixed with a fuel and ignited to created pressurized hot gas (e.g., at 3 psi). The dryer may automatically blast the heated gas with quench air to control the temperature of the heated gas before coming into contact with the microalgae. The process is then repeated multiple times to provide the pulses of heated gas. Pulse combustion heating is known to dry microalgae at a low heat which preserves the integrity and nutritional value of the microalgae. Flash drying comprises spraying or injecting a mixture of dried and undried material into a hot gas stream, and is commonly used in wastewater sludge drying.

Drying of microalgae using an incinerator or furnace may comprise heating the biomass to a high temperature (e.g., 100° C.) to evaporate the water. The heating may be performed at a level below the temperature at which the microalgae will burn and may comprise using hot gases that proceed downwardly with the biomass in parallel flow. Microalgae that are dewatered to an appropriate solids level may be dried indirectly by heating elements lining the pathway of a belt conveyor. Refractance window drying is a dehydration method that uses infra-red light, rather than high direct temperature, to remove moisture from microalgae. Wet microalgae biomass may be translated through an evaporation chamber by a belt disposed above a circulating hot water reservoir to dry the microalgae with infra-red energy in a refractance window drying. In some embodiments, microalgae dried by refractance window drying may comprise 3-8% moisture (on a wet basis).

In some embodiments, the dry composition may be mixed with water and stabilized by heating and cooling in a pasteurization process, adjustment of pH, the addition of an inhibitor of yeast and mold growth, or combinations thereof. In one non-limiting example of preparing the dried IAA enriched microalgae composition for application to plants, the microalgae harvested from the culturing system may first be held in a harvest tank before centrifuging the culture. Once the IAA enriched microalgae is centrifuged, the centrifuge discharges the fraction rich in microalgae whole cell solids, but also containing the accompanying constituents from the culture medium, into a container at a temperature of about 30° C. The IAA enriched microalgae composition may then be dried.

Techniques described herein were utilized to administer one or more of the disclosed compositions in low concentration applications. Surprisingly, this type of application was found to be effective in producing enhanced characteristics in plants. In some embodiments, a liquid composition may be administered before the seed is planted. In some embodiments, a liquid composition may be administered at the time the seed is planted. In some embodiments, a liquid composition may be administered after the seed is planted. In some embodiments, a liquid composition may be administered to plants that have emerged from the ground. In some embodiments, a dried composition may be applied to the soil before, during, or after the planting of a seed. In some embodiments, a dried composition may be applied to the soil before or after a plant emerges from the soil.

In some embodiments, the volume or mass of the IAA enriched microalgae based composition applied to a seed, seedling, or plant may not increase or decrease during the growth cycle of the plant (e.g., the amount of the microalgae composition applied to the plant does not change as the plant grows larger). In some embodiments, the volume or mass of the IAA enriched microalgae based composition applied to a seed, seedling, or plant may increase during the growth cycle of the plant (e.g., applied on a mass or volume per plant mass basis to provide more of the microalgae composition as the plant grows larger). In some embodiments, the volume or mass of the IAA enriched microalgae based composition applied to a seed, seedling, or plant may decrease during the growth cycle of the plant (e.g., applied on a mass or volume per plant mass basis to provide more of the microalgae composition as the plant grows larger).

### Seed Soak Application

In one non-limiting embodiment, the administration of the liquid composition may comprise soaking the seed in an effective amount of the liquid composition before planting the seed. In some embodiments, the administration of the liquid composition further comprises removing the seed from the liquid composition after soaking, and drying the seed before planting. In some embodiments, the seed may be soaked in the liquid composition for a time period in the range of 90-150 minutes. In some embodiments, the seed may be soaked in the liquid composition for a time period in the range of 110-130 minutes. In some embodiments, the seed may be soaked in the liquid composition for a time period in the range of 90-100 minutes. In some embodiments, the seed may be soaked in the liquid composition for a time period in the range of 100-110 minutes. In some embodiments, the seed may be soaked in the liquid composition for a time period in the range of 110-120 minutes. In some embodiments, the seed may be soaked in the liquid composition for a time period in the range of 120-130 minutes. In some embodiments, the seed may be soaked in the liquid composition for a time period in the range of 130-140 minutes. In some embodiments, the seed may be soaked in the liquid composition for a time period in the range of 140-150 minutes.

The composition may be diluted to decrease the concentration for an effective amount in a seed soak application by mixing a volume of the composition in a volume of water. The concentration of IAA enriched microalgae sourced components resulting in the diluted composition may be calculated by the multiplying the original concentration in the composition by the ratio of the volume of the composition to the volume of water. Alternatively, the grams of IAA enriched microalgae source components in the diluted composition can be calculated by the multiplying the original grams of microalgae sourced components per 100 mL by the ratio of the volume of the composition to the volume of water.

### Soil Application—Seed

In another non-limiting embodiment, the administration of the composition may comprise applying an effective amount of the composition to the soil in the immediate vicinity of the planted seed. In some embodiments, the liquid composition may be applied to the soil by injection into a low volume irrigation system, such as but not limited to a drip irrigation system supplying water beneath the soil through perforated conduits or at the soil level by fluid conduits hanging above the ground or protruding from the ground. In some embodiments, the liquid composition may be applied to the soil by a soil drench method wherein the liquid composition is poured on the soil.

The composition may be diluted to decrease the concentration to an effective amount in a soil application by mixing a volume of the composition in a volume of water. The percent solids of IAA enriched microalgae sourced components resulting in the diluted composition may be calculated by the multiplying the original concentration in the composition by the ratio of the volume of the composition to the volume of water. Alternatively, the grams of IAA enriched microalgae sourced components in the diluted composition can be calculated by multiplying the original grams of microalgae sourced components per 100 mL by the ratio of the volume of the composition to the volume of water.

The rate of application of the composition at the desired concentration may be expressed as a volume per area. In some embodiments, the rate of application of the liquid composition in a soil application may comprise a rate in the range of 50-150 gallons/acre. In some embodiments, the rate of application of the liquid composition in a soil application may comprise a rate in the range of 75-125 gallons/acre. In some embodiments, the rate of application of the liquid composition in a soil application may comprise a rate in the range of 50-75 gallons/acre. In some embodiments, the rate of application of the liquid composition in a soil application may comprise a rate in the range of 75-100 gallons/acre. In some embodiments, the rate of application of the liquid composition in a soil application may comprise a rate in the range of 100-125 gallons/acre. In some embodiments, the rate of application of the liquid composition in a soil application may comprise a rate in the range of 125-150 gallons/acre.

In some embodiments, the rate of application of the liquid composition in a soil application may comprise a rate in the range of 10-50 gallons/acre. In some embodiments, the rate of application of the liquid composition in a soil application may comprise a rate in the range of 10-20 gallons/acre. In some embodiments, the rate of application of the liquid composition in a soil application may comprise a rate in the range of 20-30 gallons/acre. In some embodiments, the rate of application of the liquid composition in a soil application may comprise a rate in the range of 30-40 gallons/acre. In some embodiments, the rate of application of the liquid composition in a soil application may comprise a rate in the range of 40-50 gallons/acre.

In some embodiments, the rate of application of the liquid composition in a soil application may comprise a rate in the range of 0.01-10 gallons/acre. In some embodiments, the rate of application of the liquid composition in a soil application may comprise a rate in the range of 0.01-0.1 gallons/acre. In some embodiments, the rate of application of the liquid composition in a soil application may comprise a rate in the range of 0.1-1.0 gallons/acre. In some embodiments, the rate of application of the liquid composition in a soil application may comprise a rate in the range of 1-2 gallons/acre. In some embodiments, the rate of application of the liquid composition in a soil application may comprise a rate in the range of 2-3 gallons/acre. In some embodiments, the rate of application of the liquid composition in a soil application may comprise a rate in the range of 3-4 gallons/acre. In some embodiments, the rate of application of the liquid composition in a soil application may comprise a rate in the range of 4-5 gallons/acre. In some embodiments, the rate of application of the liquid composition in a soil application may comprise a rate in the range of 5-10 gallons/acre.

In some embodiments, the rate of application of the liquid composition in a soil application may comprise a rate in the range of 2-20 liters/acre. In some embodiments, the rate of application of the liquid composition in a soil application may comprise a rate in the range of 3.7-15 liters/acre. In some embodiments, the rate of application of the liquid composition in a soil application may comprise a rate in the range of 2-5 liters/acre. In some embodiments, the rate of application of the liquid composition in a soil application may comprise a rate in the range of 5-10 liters/acre. In some embodiments, the rate of application of the liquid composition in a soil application may comprise a rate in the range of 10-15 liters/acre. In some embodiments, the rate of application of the liquid composition in a soil application may comprise a rate in the range of 15-20 liters/acre.

### Capillary Action Application

In another non-limiting embodiment, the administration of the liquid composition may comprise soaking a target plant seed in water, removing the seed from the water, drying the seed, applying an effective amount of the liquid composition below a seed planting level in the soil, and planting the seed. In this example, the liquid composition can be applied to the seed from the application site below, by capillary action. In some embodiments, the seed may be soaked in water for a time period in the range of 90-150 minutes. In some embodiments, the seed may be soaked in water for a time period in the range of 110-130 minutes. In some embodiments, the seed may be soaked in water for a time period in the range of 90-100 minutes. In some embodiments, the seed may be soaked in water for a time period in the range of 100-110 minutes. In some embodiments, the seed may be soaked in water for a time period in the range of 110-120 minutes. In some embodiments, the seed may be soaked in water for a time period in the range of 120-130 minutes. In some embodiments, the seed may be soaked in water for a time period in the range of 130-140 minutes. In some embodiments, the seed may be soaked in water for a time period in the range of 140-150 minutes.

The composition may be diluted to decrease the concentration for an effective amount in a capillary action application by mixing a volume of the composition in a volume of water. The concentration of microalgae sourced components resulting in the diluted composition may be calculated by the multiplying the original concentration in the composition by the ratio of the volume of the composition to the volume of water. Alternatively, the grams of IAA enriched microalgae sourced components in the diluted composition can be calculated by the multiplying the original grams of microalgae sourced components per 100 mL by the ratio of the volume of the composition to the volume of water.

### Hydroponic Application

In another non-limiting embodiment, the administration of the liquid composition to a seed, plant, or plant part may comprise applying the IAA enriched microalgae based composition in combination with a nutrient medium to seeds, plants, or plant parts disposed in a hydroponic growth medium, or an inert growth medium (e.g., coconut husks). The liquid composition may be applied initially, multiple times per day, per week, and/or per growing season.

### Foliar Application

In one non-limiting embodiment, the administration of the composition may comprise applying an effective amount of the composition to the foliage of the target plant. In some embodiments, the liquid composition may be sprayed on the foliage by a hand sprayer, a mounted sprayer, a sprayer on an agriculture implement, and/or a sprinkler.

The composition may be diluted to decrease the concentration for an effective amount in a foliar application by mixing a volume of the composition in a volume of water. The concentration of IAA enriched microalgae sourced components resulting in the diluted composition may be calculated by the multiplying the original concentration in the composition by the ratio of the volume of the composition to the volume of water. Alternatively, the grams of IAA enriched microalgae sourced components in the diluted composition can be calculated by multiplying the original grams of microalgae sourced components per 100 mL by the ratio of the volume of the composition to the volume of water.

The rate of application of the composition at the desired concentration may be expressed as a volume per area. In some embodiments, the rate of application of the liquid composition in a foliar application may comprise a rate in the range of 10-50 gallons/acre. In some embodiments, the rate of application of the liquid composition in a foliar application may comprise a rate in the range of 10-15 gallons/acre. In some embodiments, the rate of application of the liquid composition in a foliar application may comprise a rate in the range of 15-20 gallons/acre. In some embodiments, the rate of application of the liquid composition in a foliar application may comprise a rate in the range of 20-25 gallons/acre. In some embodiments, the rate of application of the liquid composition in a foliar application may comprise a rate in the range of 25-30 gallons/acre. In some embodiments, the rate of application of the liquid composition in a foliar application may comprise a rate in the range of 30-35 gallons/acre. In some embodiments, the rate of application of the liquid composition in a foliar application may comprise a rate in the range of 35-40 gallons/acre. In some embodiments, the rate of application of the liquid composition in a foliar application may comprise a rate in the range of 40-45 gallons/acre. In some embodiments, the rate of application of the liquid composition in a foliar application may comprise a rate in the range of 45-50 gallons/acre.

In some embodiments, the rate of application of the liquid composition in a foliar application may comprise a rate in the range of 0.01-10 gallons/acre. In some embodiments, the rate of application of the liquid composition in a foliar application may comprise a rate in the range of 0.01-0.1 gallons/acre. In some embodiments, the rate of application of the liquid composition in a foliar application may comprise a rate in the range of 0.1-1.0 gallons/acre. In some embodiments, the rate of application of the liquid composition in a foliar application may comprise a rate in the range of 1-2 gallons/acre. In some embodiments, the rate of application of the liquid composition in a foliar application may comprise a rate in the range of 2-3 gallons/acre. In some embodiments, the rate of application of the liquid composition in a foliar application may comprise a rate in the range of 3-4 gallons/acre. In some embodiments, the rate of application of the liquid composition in a foliar application may comprise a rate in the range of 4-5 gallons/acre. In some embodiments, the rate of application of the liquid composition in a foliar application may comprise a rate in the range of 5-10 gallons/acre.

The frequency of the application of the composition may be expressed as the number of applications per period of time (e.g., two applications per month), or by the period of time between applications (e.g., one application every 21 days). In some embodiments, the composition can be applied to the plant in a foliar application every 3-28 days. In some embodiments, the composition can be applied to the plant in a foliar application every 4-10 days. In some embodiments, the composition can be applied to the plant in a foliar application every 18-24 days. In some embodiments, the composition can be applied to the plant in a foliar application every 3-7 days. In some embodiments, the composition can be applied to the plant in a foliar application every 7-14 days. In some embodiments, the composition can be applied to the plant in a foliar application every 14-21 days. In some embodiments, the composition can be applied to the plant in a foliar application every 21-28 days.

Foliar application(s) of the composition generally begin after the plant has become established, but may begin before establishment, at defined time period after planting, or at a defined time period after emergence form the soil in some embodiments. In some embodiments, the composition can be first applied to the plant in a foliar application 5-14 days after the plant emerges from the soil. In some embodiments, the composition can be first applied to the plant in a foliar application 5-7 days after the plant emerges from the soil. In some embodiments, the composition can be first applied to the plant in a foliar application 7-10 days after the plant emerges from the soil. In some embodiments, the composition can be first applied to the plant in a foliar application 10-12 days after the plant emerges from the soil. In some embodiments, the composition can be first applied to the plant in a foliar application 12-14 days after the plant emerges from the soil.

### Soil Application—Plant

In another non-limiting embodiment, the administration of the composition may comprise applying an effective amount the composition to the soil in the immediate vicinity of the target plant. In some embodiments, the liquid composition may be supplied to the soil by injection into to a low volume irrigation system, such as but not limited to a drip irrigation system supplying water beneath the soil through perforated conduits or at the soil level by fluid conduits disposed above the ground or protruding from the ground. In some embodiments, the liquid composition may be supplied to the soil by a soil drench method wherein the liquid composition is poured on the soil.

The composition may be diluted to decrease the concentration for an effective amount in a soil application by mixing a volume of the composition in a volume of water. The concentration of IAA enriched microalgae sourced components resulting in the diluted composition may be calculated by the multiplying the original concentration of microalgae sourced components in the composition by the ratio of the volume of the composition to the volume of water. Alternatively, the grams of IAA enriched microalgae cells in the diluted composition can be calculated by multiplying the original grams of microalgae sourced components per 100 mL by the ratio of the volume of the composition to the volume of water.

The rate of application of the composition at the desired concentration may be expressed as a volume per area. In some embodiments, the rate of application of the liquid composition in a soil application may comprise a rate in the range of 50-150 gallons/acre. In some embodiments, the rate of application of the liquid composition in a soil application may comprise a rate in the range of 75-125 gallons/acre. In some embodiments, the rate of application of the liquid composition in a soil application may comprise a rate in the range of 50-75 gallons/acre. In some embodiments, the rate of application of the liquid composition in a soil application may comprise a rate in the range of 75-100 gallons/acre. In some embodiments, the rate of application of the liquid composition in a soil application may comprise a rate in the range of 100-125 gallons/acre. In some embodiments, the rate of application of the liquid composition in a soil application may comprise a rate in the range of 125-150 gallons/acre.

In some embodiments, the rate of application of the liquid composition in a soil application may comprise a rate in the range of 10-50 gallons/acre. In some embodiments, the rate of application of the liquid composition in a soil application may comprise a rate in the range of 10-20 gallons/acre. In some embodiments, the rate of application of the liquid composition in a soil application may comprise a rate in the range of 20-30 gallons/acre. In some embodiments, the rate of application of the liquid composition in a soil application may comprise a rate in the range of 30-40 gallons/acre. In some embodiments, the rate of application of the liquid composition in a soil application may comprise a rate in the range of 40-50 gallons/acre.

In some embodiments, the rate of application of the liquid composition in a soil application may comprise a rate in the range of 0.01-10 gallons/acre. In some embodiments, the rate of application of the liquid composition in a soil application may comprise a rate in the range of 0.01-0.1 gallons/acre. In some embodiments, the rate of application of the liquid composition in a soil application may comprise a rate in the range of 0.1-1.0 gallons/acre. In some embodiments, the rate of application of the liquid composition in a soil application may comprise a rate in the range of 1-2 gallons/acre. In some embodiments, the rate of application of the liquid composition in a soil application may comprise a rate in the range of 2-3 gallons/acre. In some embodiments, the rate of application of the liquid composition in a soil application may comprise a rate in the range of 3-4 gallons/acre. In some embodiments, the rate of application of the liquid composition in a soil application may comprise a rate in the range of 4-5 gallons/acre. In some embodiments, the rate of application of the liquid composition in a soil application may comprise a rate in the range of 5-10 gallons/acre.

In some embodiments, the rate of application of the liquid composition in a soil application may comprise a rate in the range of 2-20 liters/acre. In some embodiments, the rate of application of the liquid composition in a soil application may comprise a rate in the range of 3.7-15 liters/acre. In some embodiments, the rate of application of the liquid composition in a soil application may comprise a rate in the range of 2-5 liters/acre. In some embodiments, the rate of application of the liquid composition in a soil application may comprise a rate in the range of 5-10 liters/acre. In some embodiments, the rate of application of the liquid composition in a soil application may comprise a rate in the range of 10-15 liters/acre. In some embodiments, the rate of application of the liquid composition in a soil application may comprise a rate in the range of 15-20 liters/acre.

The frequency of the application of the composition may be expressed as the number of applications per period of time (e.g., two applications per month), or by the period of time between applications (e.g., one application every 21 days). In some embodiments, the composition can be applied to the plant in a soil application every 3-28 days. In some embodiments, the composition can be applied to the plant in a soil application every 4-10 days. In some embodiments, the composition can be applied to the plant in a soil application every 18-24 days. In some embodiments, the composition can be applied to the plant in a soil application every 3-7 days. In some embodiments, the composition can be applied to the plant in a soil application every 7-14 days. In some embodiments, the composition can be applied to the plant in a soil application every 14-21 days. In some embodiments, the composition can be applied to the plant in a soil application every 21-28 days.

Soil application(s) of the composition generally begin after the plant has become established, but may begin before establishment, at defined time period after planting, or at a defined time period after emergence form the soil in some embodiments. In some embodiments, the composition can be first applied to the plant in a soil application 5-14 days after the plant emerges from the soil. In some embodiments, the composition can be first applied to the plant in a soil application 5-7 days after the plant emerges from the soil. In some embodiments, the composition can be first applied to the plant in a soil application 7-10 days after the plant emerges from the soil. In some embodiments, the composition can be first applied to the plant in a soil application 10-12 days after the plant emerges from the soil. In some embodiments, the composition can be first applied to the plant in a soil application 12-14 days after the plant emerges from the soil.

Whether in a seed soak, soil, capillary action, foliar, or hydroponic application the method of use can comprise relatively low concentrations of the composition. For example, even at low concentrations, the described composition has been shown to be effective at producing an enhanced characteristic in plants. As an example, application of lower concentrations of a composition may reduce potential adverse effects impacting the environment, which can result when agricultural application are over applied to a target area. Further, the use of lower concentration can increase efficiency in the application method of use of the composition by utilizing less of the composition to produce a desired effect. In some embodiments, the use of the liquid composition with a low volume irrigation system in soil applications may allow a low concentration of the liquid composition to remain effective. That is, for example, application through the low volume irrigation system may mitigate dilution to a point where the composition is no longer capable of producing the desired effect on the plants; and may also improve water use efficiency.

In some implementations, the composition may be administered in a single application, periodically, or as needed to produce the desired affects. Surprisingly, the composition may comprise a low concentration that is administered at a low frequency of application. This type of new application technique appeared to be contrary to the traditional plant supplement application techniques. Traditionally, the concentration of an active ingredient is decreased as the frequency of application is increased to provide adequate amounts of the active ingredients. In this implementation, the effectiveness of the composition at low concentration, and fewer application frequency, appears to increase the composition's usage efficiency, while also providing the desired increase the yield efficiency of the agricultural results.

Testing has shown that the administration of a dry composition treatment to the soil, seed, or plant can be effective to produce an enhanced characteristic in the plant, when compared to a substantially identical population of an untreated plant. As an example, such enhanced characteristics can comprise: accelerated seed germination, accelerated seedling emergence, improved seedling emergence, improved leaf formation, accelerated leaf formation, improved plant maturation, accelerated plant maturation, increased plant yield, increased plant growth, increased plant quality, increased plant health, increased flowering, increased fruit yield, increased fruit growth, and/or increased fruit quality. Further, non-limiting examples of such enhanced characteristics can comprise: accelerated achievement of the hypocotyl stage, accelerated protrusion of a stem from the soil, accelerated achievement of the cotyledon stage, accelerated leaf formation, increased leaf size, increased leaf area index, increased marketable plant weight, increased marketable plant yield, increased marketable fruit weight, increased production plant weight, increased production fruit weight, increased utilization (indicator of efficiency in the agricultural process based on ratio of marketable fruit to unmarketable fruit), increased chlorophyll content (indicator of plant health), increased plant weight (indicator of plant health), increased root weight (indicator of plant health), increased root mass (indicator of plant health), increased shoot weight (indicator of plant health), increased plant height, increased thatch height, increased resistance to salt stress, increased plant resistance to heat stress (temperature stress), increased plant resistance to heavy metal stress, increased plant resistance to drought, increased plant resistance to disease improved color, reduced insect damage, reduced blossom end rot, and/or reduced sun burn. Such enhanced characteristics can occur individually in a plant, or in combinations of multiple enhanced characteristics. The characteristic of flowering may also be important for the ornamental market, and also for fruiting plants where an increase in flowering may correlate to an increase in fruit production.

### Seed Coating

In one non-limiting embodiment, the administration of the dried IAA enriched microalgae composition treatment can comprise applying the composition as a coating on a seed. In some embodiments, a seed may be coated by passing the seed through a slurry comprising IAA enriched microalgae, and then drying the coated seed. In some embodiments, the seed may be coated with the dried IAA enriched microalgae composition and other components such as, but not limited to, binders and fillers known in the art to be suitable for coating seeds. The fillers may comprise suitable inorganic particles such as, but not limited to, silicate particles, carbonate particles, and sulphate particles, quartz, zeolites, pumice, perlite, diatomaceous earth, pyrogenic silica, Sb₂O₃, TiO₂, lithopone, ZnO, and hydrated aluminum oxide. The binders may include, but are not limited to, water-soluble polymers, polyvinyl acetate, polyvinyl alcohol, polyvinyl pyrrolidone, polyurethane, methyl cellulose, carboxymethyl cellulose, hydroxylpropyl cellulose, sodium alginate, polyacrylate, casein, gelatin, pullulan, polyacrylamide, polyethylene oxide, polystyrene, styrene acrylic copolymers, styrene butadiene polymers, poly (N-vinylacetamide), waxes, carnauba wax, paraffin wax, polyethylene wax, bees wax, polypropylene wax, and ethylene vinyl acetate. In some embodiments, the seed coating may comprise a wetting and dispersing additive such as, but not limited to polyacrylates, organo-modified polyacrylates, sodium polyacrylates, polyurethanes, phosphoric acid esters, star polymers, and modified polyethers.

In some embodiments, the seed coating may comprise other components such as, but not limited to, a solvent, thickener, coloring agent, anti-foaming agent, biocide, surfactant, and/or pigment. In some embodiments, the seed coating may comprise a hydrogel or a film coating material.

In some embodiments, the concentration of dried IAA enriched microalgae in the seed coating may comprise 0.001-20% solids. In some embodiments, the concentration of IAA enriched microalgae in the seed coating may comprise less than 0.1% solids. In some embodiments, the concentration of dried IAA enriched microalgae in the seed coating may comprise 0.001-0.01% solids. In some embodiments, the concentration of dried IAA enriched microalgae in the seed coating may comprise 0.01-0.1% solids. In some embodiments, the concentration of dried IAA enriched microalgae in the seed coating may comprise 0.1-1% solids. In some embodiments, the concentration of dried IAA enriched microalgae in the seed coating may comprise 1-2% solids. In some embodiments, the concentration of dried IAA enriched microalgae in the seed coating may comprise 2-3% solids. In some embodiments, the concentration of dried IAA enriched microalgae in the seed coating may comprise 3-5% solids. In some embodiments, the concentration of dried IAA enriched microalgae in the seed coating may comprise 5-10% solids. In some embodiments, the concentration of dried IAA enriched microalgae in the seed coating may comprise 10-15% solids. In some embodiments, the concentration of dried IAA enriched microalgae in the seed coating may comprise 15-20% solids.

In some embodiments, the seed may be coated in a single step. In some embodiments, the seed may be coated in multiple steps. Conventional or otherwise suitable coating equipment or techniques may be used to coat the seeds. Suitable equipment may include drum coaters, fluidized beds, rotary coaters, side vended pan, tumble mixers, and spouted beds. Suitable techniques may comprise mixing in a container, tumbling, spraying, or immersion. After coating, the seeds may be dried or partially dried.

### Soil Application

In another non-limiting embodiment, the administration of the dried IAA enriched microalgae composition treatment can comprise mixing an effective amount of the composition with a solid growth medium, such as soil, potting mix, compost, and/or inert hydroponic material, prior to planting a seed, seedling, or plant in the solid growth medium. The dried IAA enriched microalgae composition may be mixed in the solid growth medium at an inclusion level of 0.001-20% by volume. In some embodiments, the effective amount in a mixed solid growth medium application of the dried IAA enriched microalgae composition can comprise a concentration in the range of 0.001-0.01% solids. In some embodiments, the effective amount in a mixed solid growth medium application of the dried IAA enriched microalgae composition can comprise a concentration in the range of 0.01-0.1% solids. In some embodiments, the effective amount in a mixed solid growth medium application of the dried IAA enriched microalgae composition can comprise a concentration in the range of 0.1-1% solids. In some embodiments, the effective amount in a mixed solid growth medium application of the dried IAA enriched microalgae composition can comprise a concentration in the range of 1-3%% solids. In some embodiments, the effective amount in a mixed solid growth medium application of the dried IAA enriched microalgae composition can comprise a concentration in the range of 3-5% solids. In some embodiments, the effective amount in a mixed solid growth medium application of the dried IAA enriched microalgae composition can comprise a concentration in the range of 5-10% solids. In some embodiments, the effective amount in a mixed solid growth medium application of the dried IAA enriched microalgae composition can comprise a concentration in the range of 10-20% solids.

In another non-limiting embodiment, the administration of the dried IAA enriched microalgae composition treatment can comprise inclusion in a solid growth medium during in-furrow planting or broadcast application to the ground. The dried IAA enriched microalgae composition may be applied at a rate of 50-500 grams/acre. In some embodiments, the application rate of the dried IAA enriched microalgae composition can comprise 50-100 grams/acre. In some embodiments, the application rate of the dried IAA enriched microalgae composition can comprise 100-150 grams/acre. In some embodiments, the application rate of the dried IAA enriched microalgae composition can comprise 150-200 grams/acre. In some embodiments, the application rate of the dried IAA enriched microalgae composition can comprise 200-250 grams/acre. In some embodiments, the application rate of the dried IAA enriched microalgae composition can comprise 250-300 grams/acre. In some embodiments, the application rate of the dried IAA enriched microalgae composition can comprise 300-350 grams/acre. In some embodiments, the application rate of the dried IAA enriched microalgae composition can comprise 350-400 grams/acre. In some embodiments, the application rate of the dried IAA enriched microalgae composition can comprise 400-450 grams/acre. In some embodiments, the application rate of the dried IAA enriched microalgae composition can comprise 450-500 grams/acre.

The dried IAA enriched microalgae composition may be applied at a rate of 10-50 grams/acre. In some embodiments, the application rate of the dried IAA enriched microalgae composition can comprise 10-20 grams/acre. In some embodiments, the application rate of the dried IAA enriched microalgae composition can comprise 20-30 grams/acre. In some embodiments, the application rate of the dried IAA enriched microalgae composition can comprise 30-40 grams/acre. In some embodiments, the application rate of the dried IAA enriched microalgae composition can comprise 40-50 grams/acre.

The dried IAA enriched microalgae composition may be applied at a rate of 0.001-10 grams/acre. In some embodiments, the application rate of the dried IAA enriched microalgae composition can comprise 0.001-0.01 grams/acre. In some embodiments, the application rate of the dried IAA enriched microalgae composition can comprise 0.01-0.1 grams/acre. In some embodiments, the application rate of the dried IAA enriched microalgae composition can comprise 0.1-1.0 grams/acre. In some embodiments, the application rate of the dried IAA enriched microalgae composition can comprise 1-2 grams/acre. In some embodiments, the application rate of the dried IAA enriched microalgae composition can comprise 2-3 grams/acre. In some embodiments, the application rate of the dried IAA enriched microalgae composition can comprise 3-4 grams/acre. In some embodiments, the application rate of the dried IAA enriched microalgae composition can comprise 4-5 grams/acre. In some embodiments, the application rate of the dried IAA enriched microalgae composition can comprise 5-10 grams/acre.

In some embodiments, plant treated with IAA enriched microalgae may result in an increase of at least 5% of the total fruit compared to an untreated plant and a plant treated with non-IAA enriched microalgae. In some embodiments, plant treated with IAA enriched microalgae may result in an increase of at least 10% of the total fruit fresh weight compared to an untreated plant.

### EXAMPLES

Embodiments of the techniques and systems comprising the inventive concept, described herein, are exemplified and additional embodiments are disclosed in further detail in the following Examples, which are not in any way intended to limit the scope of any aspect of the inventive concepts described herein. Analysis of the DNA sequence of the strain of Chlorella referenced in the Examples performed in the NCBI 18s rDNA reference database at the Culture Collection of Algae at the University of Cologne (CCAC) showed substantial similarity (i.e., greater than 95%) with multiple known strains of Chlorella and Micractinium. Those of skill in the art will recognize that Chlorella and Micractinium appear closely related in many taxonomic classification trees for microalgae, and strains and species may be re-classified from time to time. Thus, for references throughout the instant specification for Chlorella, it is recognized that microalgae strains in related taxonomic classifications with similar characteristics to the reference Chlorella strain would reasonably be expected to produce similar results.

### Example 1

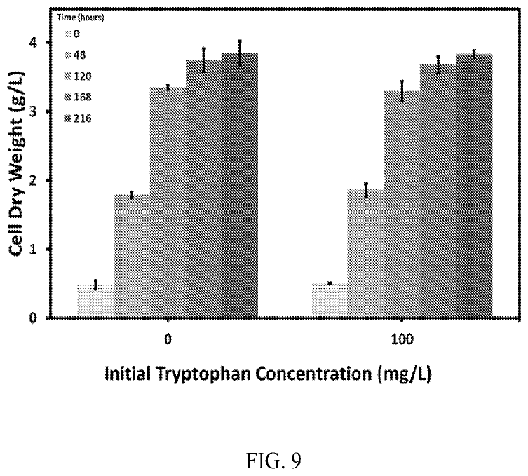

A demonstration was undertaken to illustrate that the addition of a phytohormone precursor, such as tryptophan, to a culture of microalgae can affect the growth of the microalgae in axenic conditions, and that the microalgae can convert the tryptophan to IAA. In this example, in 500 mL flask cultures, 50 mL of axenic Chlorella sp. were inoculated in 250 mL of BG-11 based culture medium and 200 mL of sterile water. For the treatments receiving tryptophan, 5 mL of sterile water was replaced with 5 mL of a 1 g/L solution of tryptophan (the equivalent of a concentration of 100 mg tryptophan/L). The cultures received 2.5 g/L of sodium acetate as an organic carbon source and 0.25 g/L NO₃ as a nitrogen source, daily. The cultures also received a supply of light to create mixotrophic culture conditions for the Chlorella. During the culturing period, samples were taken to measure dry weight at 38, 120, 168, and 216 hours. Results are shown in FIG. 9.

A shown in FIG. 9, the microalgae growth was not inhibited by the addition of 100 mg/L of tryptophan to the microalgae culture. At the end of the culturing period the biomass was analyzed for phytohormone content and compared to previous data for untreated biomass. As shown in the Table 1, the relative phytohormone concentration of the Chlorella was altered by the addition of tryptophan.

TABLE 1

| Phytohormone | Untreated biomass | Tryptophan treated biomass |
|---|---|---|
| Abscisic acid (ABA) | 39.2% | 0.0% |
| Cytokinins | 26.3% | 1.1% |
| Auxins | 31.2% | 98.9% |
| Gibberellins | 3.3% | 0.0% |

While the relative amount of auxins increased for the tryptophan treated biomass, the quantity of IAA also increased. The previously untested biomass contained 0.005-0.007 mg/L of IAA; and, by contrast, the tryptophan treated biomass contained 0.170 mg/L of unconjugated IAA, an increase of 24-34 times. In this example, these results demonstrate that the relative and absolute quantity of IAA in microalgae may be increased by treating the microalgae culture with tryptophan.

### Example 2

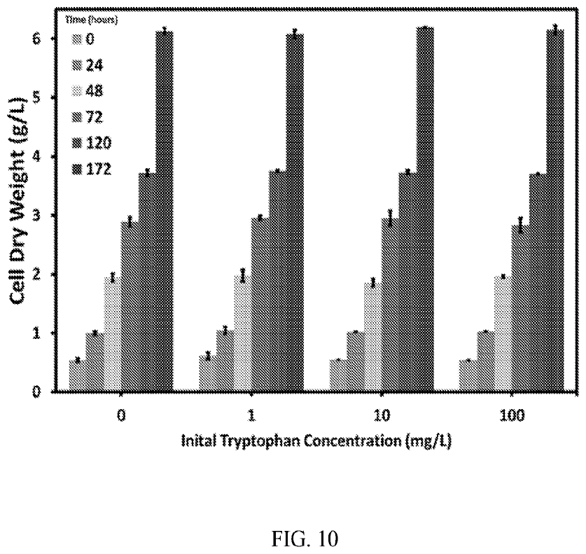

A demonstration was undertaken to illustrate the effect of different concentrations of tryptophan in a microalgae culture on the growth of the microalgae in axenic conditions; and to identify variations in the accumulation of IAA in the microalgae. In this example, in 500 mL flask cultures, axenic Chlorella sp. were inoculated in a BG-11 based culture medium. The tryptophan treatments in the microalgae cultures consisted of concentration of: 1 mg/L, 10 mg/L, and 100 mg/L. The cultures received 5 g/L of sodium acetate as an organic carbon source and 0.5 g/L NO₃ as a nitrogen source, daily. The cultures also received a supply of light to create mixotrophic culture conditions for the Chlorella. During the culturing period, samples were taken to measure dry weight at 24, 48, 72, 120, and 172 hours. Results are shown in FIG. 10.

A show in FIG. 10, the microalgae growth was not inhibited by the addition of various tryptophan treatments. At the end of the culturing period the treatment biomass was analyzed for the IAA concentration. No IAA was detected in the 1 mg/L treated biomass. 0.01 mg/L of IAA was detected in the 10 mg/L treated biomass, and 0.10 mg/L of IAA was detected in the 100 mg/L treated biomass. The 100 mg/L treated biomass was further analyzed and it was determined that 54% of the detected IAA was located in the aqueous fraction of the biomass, with the remaining IAA located in the solid fraction of the biomass. The untreated biomass was also further analyzed and it was determined that only 17% of the detected IAA was in the aqueous fraction. These results indicate that the 100 mg/L concentration of tryptophan was more effective in increasing the quantity of IAA in the microalgae cells than the 1 and 10 mg/L concentrations, and increasing the amount of IAA in the aqueous fraction.

### Example 3

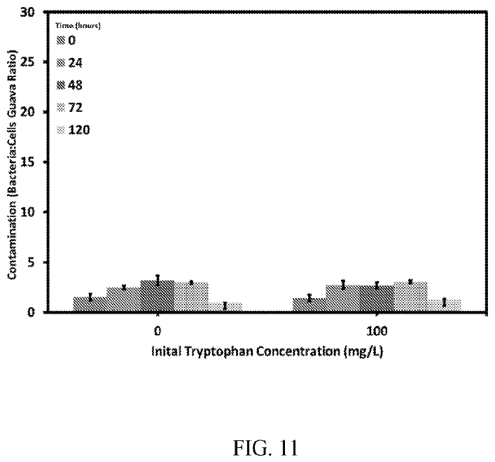

A demonstration was undertaken to illustrate the effect of treating a microalgae culture with tryptophan in non-axenic conditions effects the growth of the microalgae and increases the growth of bacteria. In this example, open, outdoor bioreactors (40 L) were inoculated with Chlorella sp. in a BG-11 based nutrient medium to produce an initial culture density of about 1 g/L in non-axenic conditions. The culture was supplied acetic acid as an organic carbon source and sodium nitrate as a nitrogen source. The culture received natural diurnal light to create mixotrophic culture conditions. The culture pH was maintained at 7.5, and the culture was supplied with air at a flow rate of 2.8 m³/hr. Half of the cultures were treated with 100 mg/L of tryptophan. Samples were taken at 24, 48, 72, 96, and 120 hours to quantify cell dry weight and the amount of bacteria. Temperature, dissolved oxygen, and pH were monitored at least two times per day. Samples were taken at the mid-point and end of the demonstration to quantify the phytohormone content. Results are shown in FIGS. 11-13.

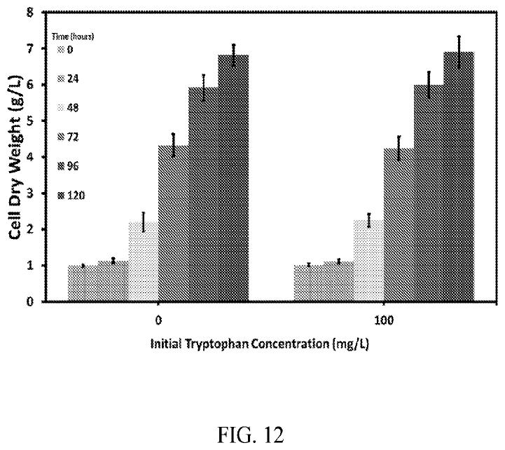

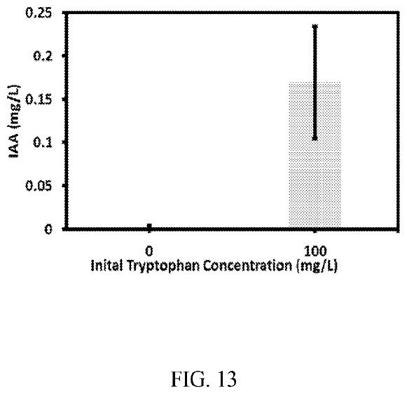

As shown in FIGS. 11-12, the cell dry-weight and bacteria:microalgae ratio was similar between treated and untreated cultures. As shown in FIG. 13, the tryptophan treated microalgae in non-axenic conditions was able to accumulate IAA. As these results indicate, the treatment of a non-axenic culture of microalgae with tryptophan did not negatively affect the growth of the microalgae or bacteria:microalgae ratio; and the microalgae successfully accumulated IAA.

### Example 4

A demonstration was undertaken to illustrate the effect of pasteurizing IAA enriched microalgae with regards to the IAA concentration. In this example, the IAA enriched Chlorella from Example 2 was subjected to a pasteurization process comprising exposing the cells to heat at about 65° C. (e.g., as described above). The microalgae contained 1,690.70 ng IAA/g Fresh Weight (FW) prior to pasteurization, and 1,518.84 ng IAA/g FW after pasteurization. The results indicated that the pasteurization process only decreased the amount of IAA in the microalgae by 10.2%. Therefore, pasteurization of the IAA enriched microalgae may be effective for use in product compositions without substantially degradation of the IAA content.

### Example 5

A demonstration was undertaken to illustrate the effect of application of IAA enriched microalgae to plants, when compared to non-IAA enriched microalgae that are applied to plants. In this demonstration, pasteurized microalgae treatments consisting of 10% solids of mixotrophically culture Chlorella sp. or the IAA enriched Chlorella sp. produced in Example 4 were prepared for application to bell pepper seedlings, and compared to an untreated control. All plants received the same nitrogen-phosphorus-potassium (NPK) feeding regime. Thirty bell pepper seedlings for each treatment were germinated in coconut coir, transplanted to quart sized pots filled with Turface (a calcined clay substrate), and attached to a drip-to-waste hydroponic system. Growing conditions for the plants were controlled at about 23° C., 45% relative humidity, and ambient light at 200 μmol PAR. The plants were fertigated with a basal solution of vegetative nutrients along with microalgae treatment aliquots added at a concentration of 9 mL/gal. Based on the stage of growth, plants were dosed with the microalgae treatment solutions for 10 or 20 minutes three times per day. A portion of the plants were harvested at 21 days and 42 days after transplanting to collect vegetative and early reproductive data. The final harvest for data collection occurred at 62 days. Differences among the treatments were evaluated using analysis of variance (ANOVA) followed by a means comparison using Tukey's Honestly Significant Differences (THSD).

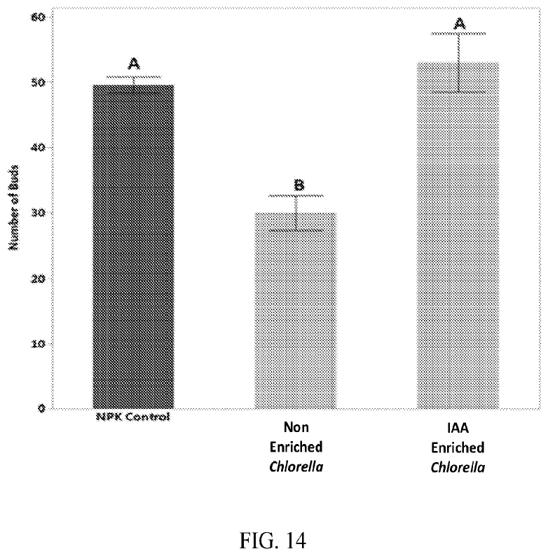

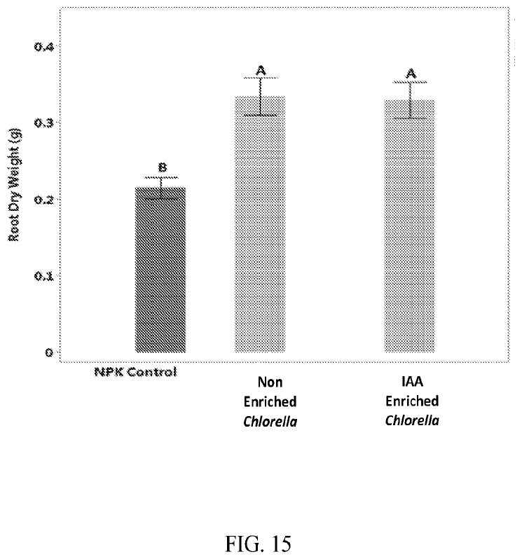

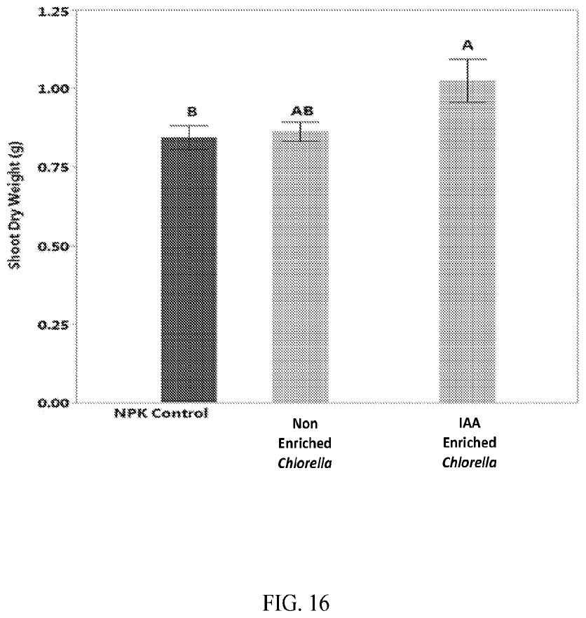

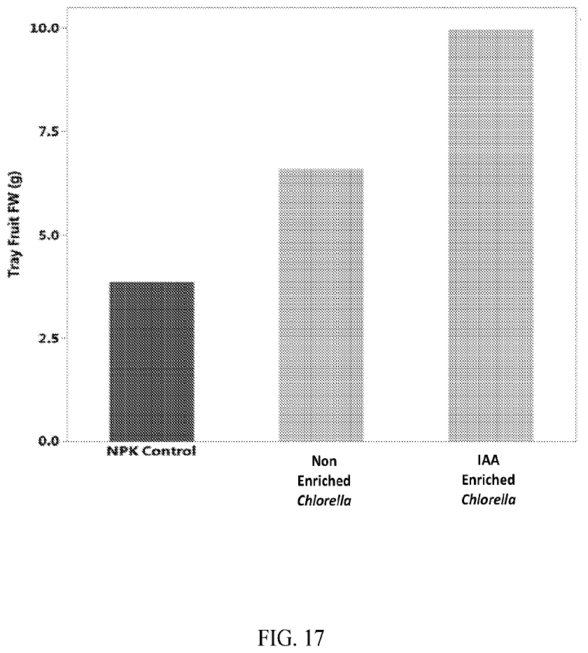

Fresh weight of the plant was calculated as the sum of root fresh weight and shoot fresh weight. Shoot fresh weight was determined by cutting the stem of the plant at the point where the plant emerges from the soil, removing any peppers which may have grown in the shoot material, and weighing the biomass on an analytical balance. Root fresh weight was determined by shaking excess soil off the root ball, washing gently with city water, blotting dry with paper towels, and then weighing. Tray fruit fresh weight was measured by cutting peppers off all of the plants for a treatment and weighted together. Dry weight was calculated as the sum of root dry weight and shoot dry weight. After the fresh weight was measured, the roots and shoots were folded up into individual paper bags and left in a drying oven set to 75° C. for 5 days before being weighed. At the time of harvest the number of buds, flowers, and peppers were counted for each plant. The results are show in Tables 2-4 for all data collected, and FIGS. 14-17 for the day 42 data which also indicate the statistical significance with an alpha identifier (i.e., A, B, AB) and variation with an error bar.

TABLE 2

| | Day 21 | | |
|---|---|---|---|
| | IAA | % difference | % difference |
| | Enriched | from non- | from |
| | Chlorella | enriched | untreated |
| | (Average) | Chlorella | control |
| Circumference (cm) | 76.6 | +11.1 | +2.4 |
| Height (cm) | 7.3 | +10.6 | −3.3 |
| Fresh Weight (g) | 14.4 | +17.3 | +9.1 |
| Root Fresh Weight (g) | 5.1 | +9.6 | +0.7 |
| Shoot Fresh Weight (g) | 9.3 | +21.9 | +14.2 |
| Dry Weight (g) | 1.4 | +13.0 | +28.0 |
| Root Dry Weight (g) | 0.3 | −1.5 | +53.5 |
| Shoot Dry Weight (g) | 1.0 | +18.7 | +21.5 |
| Number of Buds | 5.5 | +39.2 | +29.4 |

TABLE 3

| | Day 42 | | |
|---|---|---|---|
| | | % difference | % difference |
| | IAA | from non- | from |
| | Enriched | enriched | untreated |
| | Chlorella | Chlorella | control |
| Avg. Circumference (cm) | 128.0 | +17.8 | −3.5 |
| Avg. Height (cm) | 22.1 | +20.7 | −22.6 |
| Avg. Fresh Weight (g) | 174.2 | +30.1 | −4.4 |
| Avg. Root Fresh Weight (g) | 69.4 | +21.1 | +12.1 |
| Avg. Shoot Fresh Weight (g) | 104.8 | +36.8 | −12.8 |
| Avg. Dry Weight (g) | 15.2 | +22.7 | +1.8 |
| Avg. Root Dry Weight (g) | 5.1 | +21.3 | +10.8 |
| Avg. Shoot Dry Weight (g) | 10.1 | +23.4 | −2.3 |
| Avg. Number of Buds | 53.0 | +76.7 | +6.9 |
| Avg. Number of Flowers | 6.2 | +93.8 | +40.9 |
| Avg. Weight/Pepper (g) | 1.4 | +51.1 | −26.1 |
| Tray Pepper Count | 7 | 0.0 | +250.0 |
| Tray Fruit Fresh Weight (g) | 9.96 | +51.1 | +158.7 |
| Tray Pepper Dry Weight (g) | 0.72 | +26.3 | +100.0 |

TABLE 4

| | Day 62 | | |
|---|---|---|---|
| | | % difference | % difference |
| | IAA | from non- | from |
| | Enriched | enriched | untreated |
| | Chlorella | Chlorella | control |
| Avg. Circumference (cm) | 171.8 | −2.0 | −11.1 |
| Avg. Height (cm) | 32.7 | −4.1 | −23.4 |
| Avg. Fresh Weight (g) | 376.7 | −7.5 | −23.4 |
| Avg. Root Fresh Weight (g) | 171.7 | −4.0 | −26.7 |
| Avg. Shoot Fresh Weight (g) | 205.0 | −10.2 | −20.5 |
| Avg. Dry Weight (g) | 42.8 | +5.1 | −29.2 |
| Avg. Root Dry Weight (g) | 18.9 | +28.2 | −38.2 |
| Avg. Shoot Dry Weight (g) | 23.9 | −7.9 | −20.0 |
| Total Number of Peppers | 48 | +23.1 | +6.7 |
| Avg. Weight/Pepper (g) | 17.24 | −15.8 | +12.2 |
| Tray Fruit Fresh Weight (g) | 827.63 | +3.7 | +19.7 |

### Aspects of the Inventive Concept

In one non-limiting embodiment, a method may comprise: inoculating a culture, comprising microalgae cells in an aqueous culture medium, with at least 50 mg/L of tryptophan; culturing the microalgae with a source of carbon; resulting in production of a concentration of Indole-3-acetic acid (IAA) in the microalgae cells in the range of 0.01-1.0 mg IAA/L. In some embodiments, the source of carbon may comprise at least one of acetate and acetic acid. In some embodiments, the microalgae may be Chlorella.

In some embodiments, the amount of tryptophan may be in the range of 50-500 mg/L. In some embodiments, the amount of tryptophan may be in the range of 100-200 mg/L. In some embodiments, the concentration of IAA in the resulting microalgae cells may be in the range of 0.10-0.20 mg IAA/L.

In another non-limiting embodiment, a method may comprise: inoculating a culture, comprising microalgae cells in an aqueous culture medium, with a precursor of at least one phytohormone; culturing the microalgae; and increasing the concentration of the at least one phytohormone in the microalgae cells in the range of 100-5,000%. In some embodiments, the precursor may be tryptophan and the at least one phytohormone may be Indole-3-acetic acid.

In some embodiments, the microalgae may be cultured in phototrophic conditions. In some embodiments, the microalgae may be cultured in mixotrophic conditions. In some embodiments, the microalgae may be cultured in heterotrophic conditions. In some embodiments, the concentration of the at least one phytohormone in the aqueous fraction may be increased to at least 50%.

In another non-limiting embodiment, a method may comprise: enriching a culture of microalgae cells with a first concentration of at least one phytohormone in the cells; and pasteurizing the culture of microalgae cells to produce a second concentration of at least one phytohormone in the cells, wherein the second concentration comprises a decrease of less than 30% from the first concentration.

In another non-limiting embodiment, a method of plant enhancement may comprise administering a composition treatment to a plant, seedling, or seed, where the composition treatment comprises 0.001-0.1% by weight of microalgae whole biomass, and comprises a concentration in the range of 0.10-0.20 mg IAA/L to enhance at least one plant characteristic. In some embodiments, the composition treatment may be applied at a rate of 5-15 mL/gal. In some embodiments, the microalgae may be Chlorella. It is to be appreciated that the microalgae whole biomass can comprise a concentration of at least 0.10 mg IAA/L and such concentration can be selected with sound engineering judgement without departing from the scope of the subject innovation. For instance, the subject innovation provides ranges for mg IAA/L concentrations but are for example and not to be limiting on the subject innovation. By way of example and not limitation, the microalgae whole biomass can comprise a concentration in the range of 0.10-0.20 mg IAA/L. In another example, the microalgae whole biomass can comprise a concentration in a range of 0.10—X mg IAA/L, where X is greater than 0.20.

In another non-limiting embodiment, a composition may comprise whole microalgae cells with a concentration in the range of 0.10-0.20 mg IAA/L. In some embodiments, the whole microalgae cells may be pasteurized. In some embodiments, at least 30% of the IAA in the whole microalgae cells may be located in the aqueous fraction. In some embodiments, microalgae may be Chlorella. In some embodiments, the composition may further comprise at least one of water and soil.

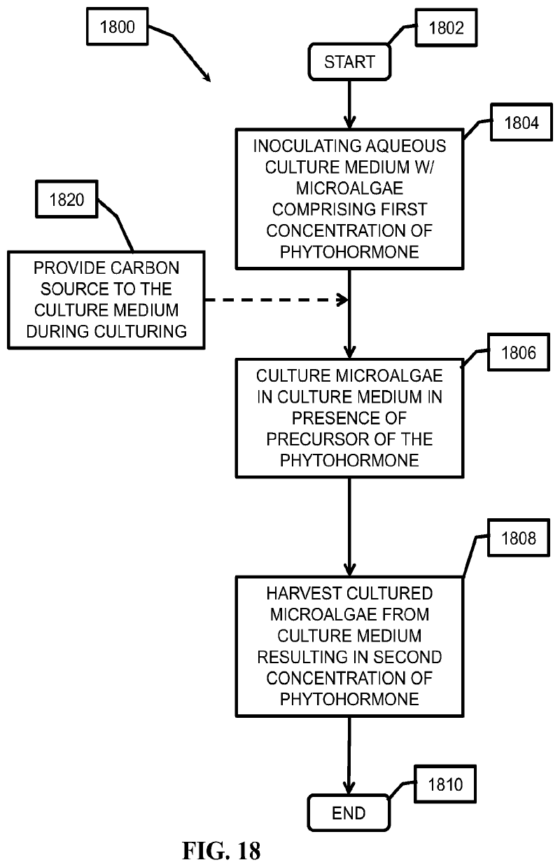

A method may be devised and used for increasing a phytohormone yield in a microalgal culture. FIG. 18 is flow diagram illustrating an exemplary method 1800 for increasing a phytohormone yield in a microalgal culture. In this implementation, the exemplary method 1800 begins at 1802. At 1804, an aqueous culture medium can be inoculated with microalgae cells that comprise a first concentration of at least one phytohormone in the microalgae cells. At 1806, the microalgae cells can be cultured in the culture medium in the presence of a precursor of the at least one phytohormone. At 1808, the cultured microalgae cells can be harvested from the culture medium, resulting in a second concentration of the at least one phytohormone in the microalgae cells. In this exemplary method, the second concentration comprises an increase in concentration by an amount in a range of 50%-5,000% from the first concentration. Having harvested the microalgae cells, the exemplary method 1800 ends at 1810. In one implementation, as further illustrated in FIG. 18, at 1820, a source of carbon can be provided to the culture medium during culturing of the microalgae cells.

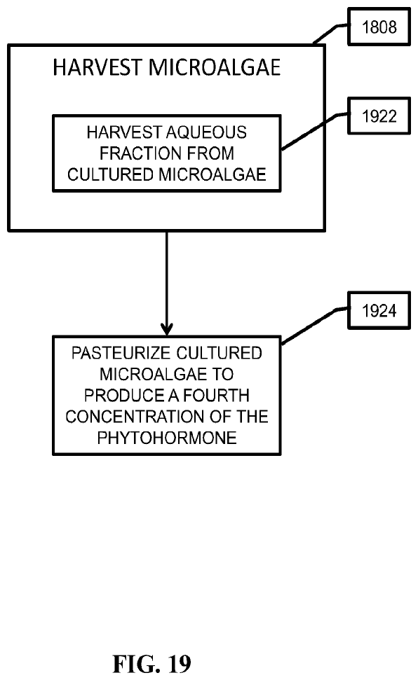

In one implementation, as illustrated in FIG. 19, the harvesting of the cultured microalgae cells from the culture medium 1808, can further comprise harvesting an aqueous fraction from the cultured microalgae cells, at 1922. In this example, the aqueous fraction can comprise a third concentration of the at least one phytohormone, wherein the third concentration comprises an increase by at least fifty percent (50%) from the first concentration. In one implementation, as illustrated in FIG. 19, the exemplary method 1800 may comprise pasteurizing the cultured microalgae cells to produce a fourth concentration of the at least one phytohormone in the cells, at 1924, wherein the fourth concentration comprises a decrease of less than 30% from the second concentration.

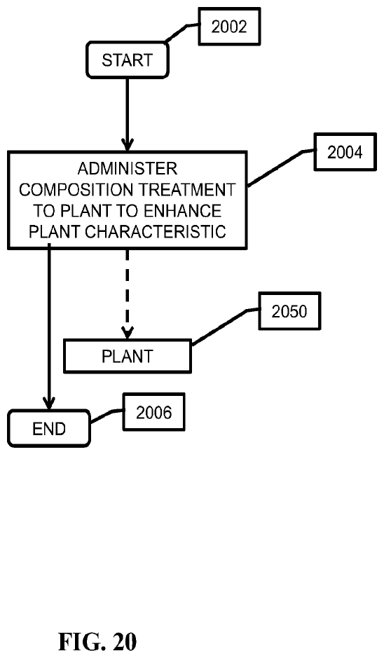

A method may be devised for plant enhancement. FIG. 20 is a diagram that illustrates an exemplary method 2000 for plant enhancement. This exemplary method 2000 begins at 2002. At 2004, a composition treatment can be administered to a plant, a portion of a plant, a seedling, or seed 2050 a composition in order to enhance at least one plant characteristic. In this exemplary method, the composition treatment can comprise 0.001-0.1% by weight of microalgae whole biomass; and the microalgae whole biomass can comprise a concentration of at least 0.10 mg IAA/L. In another exemplary method, the composition treatment can comprise 0.001-0.1% by weight of microalgae whole biomass; and the microalgae whole biomass can comprise a concentration in the range of 0.10-0.20 mg IAA/L. Having administered the composition treatment to the plant 2050, the exemplary method 2000 ends at 2006.

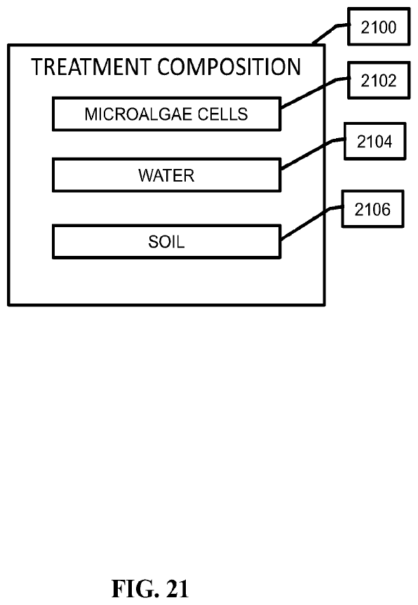

A composition may be devised for treating plant material, in order to enhance at least one plant characteristic. FIG. 21 is a diagram that illustrates a composition 2100 that may be used for treating plant material to enhance at least one plant characteristic. In this implementation, the example composition 2100 can comprise whole microalgae cells 2102 that comprise a concentration in the range of 0.10-0.20 mg IAA/L in the microalgae cells. In one implementation, the composition treatment may further comprise water 2104. In one implementation, the composition treatment may further comprise soil 2106.

All references, including publications, patent applications, and patents, cited herein, are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All provided ranges of values are intended to include the end points of the ranges, as well as values between the end points.

The citation and incorporation of patent documents herein is done for convenience only and does not reflect any view of the validity, patentability, and/or enforceability of such patent documents.

The inventive concepts described herein include all modifications and equivalents of the subject matter recited in the claims and/or aspects appended hereto as permitted by applicable law.

### REFERENCES

Bar, Tami, and Yaacov Okon. “Tryptophan conversion to indole-3-acetic acid via indole-3-acetamide in Azospirillum brasilense Sp7.” Canadian journal of microbiology 39.1 (1993): 81-86.
Kosuge, T., M. G. Heskett, and E. E. Wilson. “Microbial synthesis and degradation of indole-3-acetic acid I. The conversion of L-tryptophan to indole-3-acetamide by an enzyme system from Pseudomonas savastanoi.” Journal of Biological Chemistry 241.16 (1966): 3738-3744.
Won C, Shen X, Mashiguchi K, et al. Conversion of tryptophan to indole-3-acetic acid by TRYPTOPHAN AMINOTRANSFERASES OF ARABIDOPSIS and YUCCAS in Arabidopsis. Proc Natl Acad Sci. 2011; 108(45):18518-18523. doi:10.1073/pnas.1108436108.
Spaepen S et al. (2007) Indole-3-acetic acid in microbial and microorganism-plant signaling. Federation of European Microbiological Societies Microbiology Reviews 31: 425-448.
San-Francisco, S., Houdusse, F., Zamarreño, A. M., Garnica, M., Casanova, E., & Garcia-Mina, J. M. (2005). Effects of IAA and IAA precursors on the development, mineral nutrition, IAA content and free polyamine content of pepper plants cultivated in hydroponic conditions. Scientia Horticulturae, 106(1), 38-52.
Liu, et al. Stimulatory effect of auxins on growth and lipid productivity of Chlorella pyrenoidosa and Scenedesmus quadricauda. Algal Research, Volume 18, September 2016, p. 273-280.
Jusoh, et al. Indole-3-acetic acid (IAA) induced changes in oil content, fatty acid profiles and expression of four fatty acid biosynthetic genes in Chlorella vulgaris at early stationary growth phase. Phytochemistry, Volume 111, March 2015, pages 65-71.
Bajguz, et al. Synergistic effect of auxins and brassinosteroids on the growth and regulation of metabolite content in the green algal Chlorella vulgaris (Trebouxiophyceae). Plant Physiology and Biochemistry, Volume 71, October 2013, p. 290-297.
Lau, et al. Auxin signaling in algal lineages: fact or myth? Trends in Plant Science, Volume 14, Issue 4, April 2009, Pages 182-188.
U.S. Patent Publication No. 2015/0004704
U.S. Patent Publication No. 2010/0162620
U.S. Patent Publication No. 2011/0091945
W.I.P.O. Patent Publication No. 2014060973A1
U.S. Patent Publication No. 2014/0093922

Although a particular feature of the disclosed techniques and systems may have been disclosed with respect to only one of several implementations, such feature may be combined with one or more other features of the other implementations as may be desired and advantageous for any given or particular application. Also, to the extent that the terms “including”, “includes”, “having”, “has”, “with”, or variants thereof are used in the detailed description and/or in the claims, such terms are intended to be inclusive in a manner similar to the term “comprising.”

This written description uses examples to disclose the inventive concepts, including the best mode, and also to enable one of ordinary skill in the art to practice the innovations, including making and using any devices or systems and performing any incorporated methods. The patentable scope of the inventive concepts is defined by the claims, and may include other examples that occur to those skilled in the art. Such other examples are intended to be within the scope of the claims if they have structural elements that are not different from the literal language of the claims, or if they include equivalent structural elements with insubstantial differences from the literal language of the claims.

In the specification and claims, reference will be made to a number of terms that have the following meanings. The singular forms “a”, “an” and “the” include plural referents unless the context clearly dictates otherwise. Approximating language, as used herein throughout the specification and claims, may be applied to modify a quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Moreover, unless specifically stated otherwise, a use of the terms “first,” “second,” etc., do not denote an order or importance, but rather the terms “first,” “second,” etc., are used to distinguish one element from another.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

The best mode for carrying out the inventive concepts has been described for purposes of illustrating the best mode known to the applicant at the time and enable one of ordinary skill in the art to practice the innovations, including making and using devices or systems and performing incorporated methods. The examples are illustrative only and not meant to limit the inventive concepts disclosed, as measured by the scope and merit of the claims. The inventive concepts disclosed have been described with reference to preferred and alternate embodiments. Obviously, modifications and alterations will occur to others upon the reading and understanding of the specification. It is intended to include all such modifications and alterations insofar as they come within the scope of the appended claims or the equivalents thereof. The patentable scope of the inventive concepts disclosed is defined by the claims, and may include other examples that occur to one of ordinary skill in the art. Such other examples are intended to be within the scope of the claims if they have structural elements that do not differentiate from the literal language of the claims, or if they include equivalent structural elements with insubstantial differences from the literal language of the claims.

1. A method for increasing a phytohormone yield in a microalgal culture, comprising:
inoculating an aqueous culture medium with microalgae cells comprising a first concentration of at least one phytohormone in the microalgae cells;
culturing the microalgae cells in the culture medium in the presence of a precursor of the at least one phytohormone; and
harvesting cultured microalgae cells from the culture medium resulting in a second concentration of the at least one phytohormone in the microalgae cells, wherein the second concentration comprises an increase in concentration by an amount in a range of 50%-5,000% from the first concentration.

2. The method of claim 1, further comprising providing a source of carbon to the culture medium during culturing of the microalgae cells.

3. The method of claim 2, wherein the source of carbon comprises at least one of acetate and acetic acid.

4. The method of claim 1, wherein the precursor is tryptophan and the at least one phytohormone is Indole-3-acetic acid.

5. The method of claim 4, wherein the concentration of tryptophan is at least 10 mg/L.

6. The method of claim 4, wherein the concentration of Indole-3-acetic acid (IAA) in the cultured microalgae cells is in the range of 0.01-1.0 mg IAA/L.

7. The method of claim 5, wherein the amount of tryptophan is in the range of 10-500 mg/L.

8. The method of claim 7, wherein the amount of tryptophan is in the range of 100-200 mg/L.

9. The method of claim 6, wherein the concentration of IAA in the microalgae cells is in the range of 0.10-0.20 mg IAA/L.

10. The method of claim 1, wherein the microalgae is Chlorella.

11. The method of claim 1, wherein the microalgae is cultured in phototrophic conditions.

12. The method of claim 1, wherein the microalgae is cultured in mixotrophic conditions.

13. The method of claim 1, wherein the microalgae is cultured in heterotrophic conditions.

14. The method of claim 1, further comprising harvesting an aqueous fraction from the cultured microalgae cells, wherein the aqueous fraction comprising a third concentration of the at least one phytohormone, wherein the third concentration comprises an increase by at least 50% from the first concentration.

15. The method of claim 1, further comprising pasteurizing the cultured microalgae cells to produce a fourth concentration of the at least one phytohormone in the cells, wherein the fourth concentration comprises a decrease of less than 30% from the second concentration.

16. A method of enhancing growth of a plant comprising administering an effective amount of a liquid composition treatment comprising phytohormone-enriched Chlorella to the plant, the composition comprising pasteurized phytohormone-enriched Chlorella cells.

17. The method of claim 16, wherein the phytohormone is Indole-3-acetic acid (IAA) and the concentration of IAA in the cultured microalgae cells is in the range of 0.01-1.0 mg IAA/L.

18. The method of claim 16, wherein the microalgae is Chlorella.

19. A composition for treating plant material to enhance at least one plant characteristic, comprising:
whole pasteurized microalgae cells comprising a concentration in the range of 0.01-1.0 mg IAA/L in the microalgae cells.

20. (canceled)

21. The composition of claim 19, wherein at least 30% of the IAA in the whole microalgae cells is located in an aqueous fraction.

22. (canceled)
